(19) United States
(12) Patent Application Publication
(10) Pub. No.: US 2019/0135924 A1
(43) Pub. Date: May 9, 2019

(54) ANTI-HLA MONOCLONAL CHIMERIC IMMUNOGLOBULIN, PROCESS AND KIT EMPLOYING SUCH A MONOCLONAL CHIMERIC IMMUNOGLOBULIN

(71) Applicants: UNIVERSITE PAUL SABATIER TOULOUSE III, Toulouse (FR); CENTRE HOSPITALIER UNIVERSITAIRE DE TOULOUSE, Toulouse Cedex (FR); INVIVOGEN, Toulouse (FR)

(72) Inventors: Antoine Blancher, Toulouse (FR); Nicolas Congy, La Croix Falgarde (FR); Jean-Gerard Tiraby, Toulouse (FR); Daniel Drocourt, Saint Orens De Gameville (FR)

(21) Appl. No.: 16/176,472

(22) Filed: Oct. 31, 2018

Related U.S. Application Data
(63) Continuation-in-part of application No. 14/379,048, filed on Aug. 15, 2014.

(30) Foreign Application Priority Data
Feb. 16, 2012 (FR) 1200450

(51) Int. Cl. C07K 16/28; G01N 33/68; G01N 33/52; G01N 33/543

(52) U.S. Cl. CPC C07K 16/2833; G01N 33/6803; G01N 33/52; G01N 2333/70539; C07K 2317/51; C07K 2317/515; G01N 33/543

(57) ABSTRACT

A method for determining the quantity of anti-HLA antibodies of a liquid medium containing antibodies, including determining a calibration curve defined by a plurality of measured values corresponding to a determined quantity of a monoclonal chimeric immunoglobulin, measuring the value corresponding to the liquid medium, and deducing the quantity of anti-HLA antibody in the liquid medium from the calibration curve and from the measured value corresponding to the liquid medium.

The invention relates to a process for determining the quantity of anti-HLA antibodies in a liquid medium containing antibodies. The invention relates also to an anti-HLA class I or anti-HLA class II monoclonal chimeric immunoglobulin for carrying out such a process. The invention relates in particular to such a monoclonal chimeric immunoglobulin which is suitable for use especially as a standardization reagent for the screening and quantification of anti-HLA antibodies in a liquid medium, especially in a biological liquid medium. The invention relates in particular to such a monoclonal chimeric immunoglobulin having on the one hand the function of a monoclonal antibody and on the other hand a chimeric structure. The invention relates also to a standardized process for the screening of anti-HLA antibodies in a liquid medium and to a process for the quantification of anti-HLA antibodies in a liquid medium, in which processes such a monoclonal chimeric immunoglobulin is used. In particular, the invention relates to such a process for the quantification of anti-HLA antibodies in the serum of a patient, especially of a transplant patient or a patient awaiting a transplant.

The invention relates further to a diagnostic kit for carrying out such a process. In particular, the invention relates to such a process and such a diagnostic kit which are suitable for permitting accurate, reliable and rapid quantification of anti-HLA antibodies in a liquid medium, especially in a biological fluid collected from a patient.

In the field of organ transplantation, it has been known since the 1930s that compatibility between the donor's tissue type, as defined by the HLA (Human Leucocyte Antigen) antigens, and the recipient's immune system, especially the antibodies, is essential to the success of the organ transplant.

The HLA antigens are carried by two types of membrane proteins which are highly immunogenic: HLA class I molecules and HLA class II molecules. Accordingly, the exposure of an individual to HLA alloantigens, that is to say antigens that are foreign and different from his own, can lead to the development of an immune response to those antigens. This immune response can be cell-mediated (alloreactive T lymphocytes) or humoral (synthesis of anti-HLA antibodies).

HLA class I antigens are coded for by three genes HLA-A, HLA-B and HLA-C, the polymorphism of which is responsible for the three series of alleles HLA-A, HLA-B and HLA-C, respectively. HLA class II antigens are coded for by the genes HLA-DP, HLA-DQ and HLA-DR.

In organ transplantation, it is crucial to minimize—or even eliminate—the risks of proposing to a patient awaiting a transplant an organ for transplant that expresses HLA antigens against which the patient is already immunized. In this situation, the risk of the occurrence of hyperacute humoral rejection—that is to say humoral rejection within a period of less than 24 hours following the transplant—is considerable. In addition, within the context of transplant monitoring, the early screening of the appearance in the transplant recipient patient of antibodies directed against the antigens of the transplanted organ allows said transplant recipient patient to be treated as early as possible in an attempt to control the development of the humoral response, which may result in the destruction of the transplant.

Monitoring of the alloimmunization of both transplant patients and patients awaiting a transplant is therefore essential in order to ensure the survival of the transplants and of the transplant patients.

Within this context, it is necessary to be able to detect, identify and quantify anti-HLA antibodies in patients awaiting a transplant and in transplant patients. Numerous techniques for detecting anti-HLA antibodies have already been developed.

There is known in particular the technique called “complement-dependent microlymphocytotoxicity”. This technique consists in presenting the serum of a patient, especially of a transplant recipient, to a series of cells of known HLA typing in the presence of rabbit complement. If antibodies (Ab) specific to the HLA antigens carried by the cells are present in the tested serum, and if those antibodies are capable of activating the complement (antibodies of class IgM and of subclass IgG-1 and IgG-3), complement-dependent cell lysis (CDC, Complement Dependent Cytotoxicity) reveals the presence of the antibodies. By virtue of a panel of cells expressing different HLA antigens, it is thus possible to screen the antibodies and then identify their specificity/specificities. This reference technique permits the detection of cytolytic anti-HLA antibodies, which are the most dangerous for the transplanted organ. However, this technique has low sensitivity in comparison with more recent techniques. This technique, which requires either the availability of a large variety of lymphocytes from donors of known HLA phenotype or the in vitro cultivation of a large number of HLA-typed cell lines, is therefore complex and laborious to carry out.

More sensitive techniques are also known, such as immunoenzymatic assay on a solid substrate (“ELISA” for “Enzyme-Linked ImmunoSorbent Assay”). In addition, there has recently appeared the technique of immunofluorimetry coupled with detection in flow, which is designed on the principle of flow cytometry. The principle of flow immunofluorimetry consists in fixing purified HLA class I or

HLA class II antigens to the surface of polystyrene beads. The anti-HLA antibodies which recognize the HLA class I or HLA class II antigens bind to the antigens bound to the surface of the beads and are revealed by anti-IgG secondary antibodies coupled to a fluorescent group after washing of the polystyrene beads. The secondary antibodies are detected by flow fluorimetry. Their fluorescence intensity is additionally quantified.

For screening tests there is used a plurality of types of beads in admixture, each type of beads carrying on the surface a plurality of HLA antigens, either of class I or of class II. Such an approach allows the presence of anti-HLA antibodies to be detected but without permitting the identification of their specificity/specificities.

In order to identify and characterize the specificity of the antibody, on the other hand, there is used a plurality of types of beads in admixture, each type of beads carrying on the surface a single HLA antigen.

Kits for the detection and identification of anti-HLA antibodies are known. They comprise polystyrene beads coated with HLA class I antigens or HLA class II antigens, and polystyrene beads coated with human IgGs. Also marketed are an anti-human IgG secondary antibody coupled to phycoerythrin, and a serum without anti-HLA antibodies as negative control. Such a negative control is suitable for quantifying the non-specific fixing of the secondary antibody to the polystyrene beads. Such kits do not comprise a positive control, or a sensitivity control, or a standard allowing the concentration of anti-HLA antibodies (expressed, for example, in mole/l or in g/l) in the analyzed medium to be derived precisely from the measured fluorescence intensity.

In addition, such kits without a calibration and/or sensitivity control do not allow the sensitivity threshold of the analysis method to be determined, that is to say the minimum value of the signal that makes it possible to affirm that the signal observed is significantly greater than the background noise of the measurement.

In order to remedy this lack of a positive control in methods for the screening and/or quantification of anti-HLA antibodies, immunology and histocompatibility laboratories use, as positive control, a mixture of several serums of several individuals immunized against several HLA antigens.

In such a positive control, the concentration of each of the antibodies of the serums of the immunized individuals is unknown. Such a mixture of serums does not allow the intensity of the fluorescence measurement to be correlated with a concentration (mol/l or g/l) of a specific antibody of the mixture of serums. It therefore does not allow the antibodies present in the serum of the transplant patient or patient awaiting transplant to be quantified. It therefore also does not allow the real risks of the occurrence of a hyperacute humoral response to be evaluated.

The reactivity of such a mixture of serums is variable from one antigen to another, and their use does not allow the detection threshold to be fixed for each HLA antigen studied. Moreover, such a mixture of polyclonal antibodies obtained from patient serums is available in a limited quantity and is quickly exhausted. It must therefore be replaced by a different mixture, which is also available in a variable quantity, which does not allow said mixture to be exchanged between laboratories with a view to standardization of the results. The variability of the mixtures of serums from one batch to another requires frequent validation of the batches, which are neither comparable nor reproducible from one batch to another.

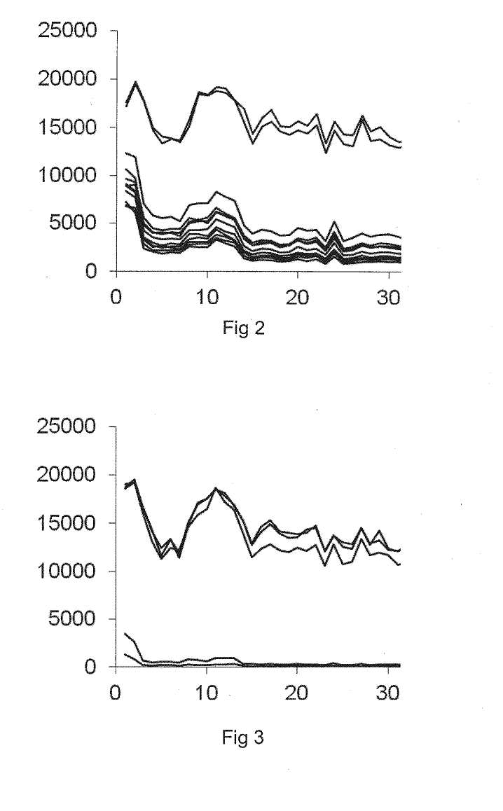

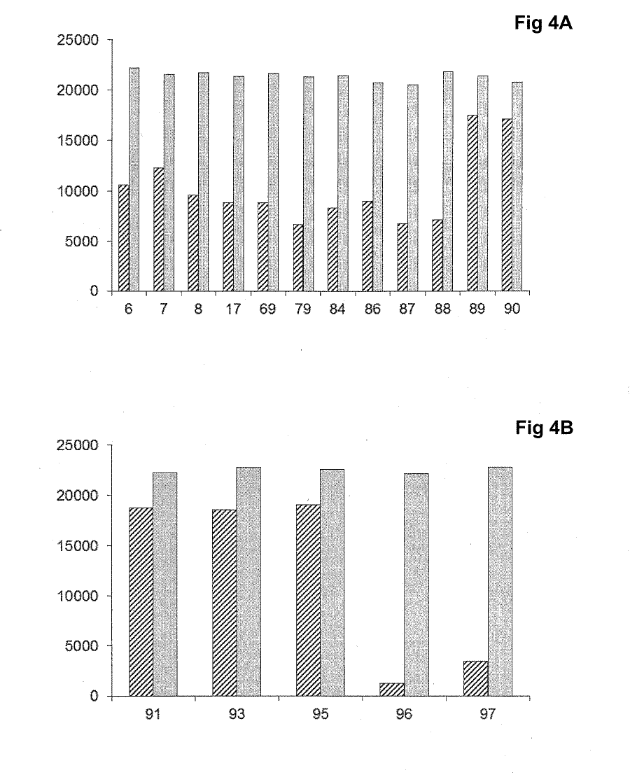

Using such a mixture of serums, the inventors have shown (FIGS. 2, 3 and 4) that the fluorescence intensity value associated with each type of polystyrene beads depends on the nature of the HLA class I or class II antigen carried by each of the types of polystyrene beads. In addition, the fluorescence intensity value associated with each of the types of polystyrene beads shows considerable variability over time, especially over a period of approximately five months. That value varies (FIG. 4A and FIG. 4B, hatched histograms) between 1000 and 20,000 average fluorescence units.

As a result, the average value of the fluorescence intensity measured on all the polystyrene beads exhibits a considerable dispersion (calculated by its standard deviation), which does not allow the anti-HLA antibodies in the liquid medium to be quantified. Such a dispersion is shown in FIG. 4A and FIG. 4B (hatched histograms) of the present patent application, which is given to illustrate a standard of the prior art.

Such a dispersion of the fluorescence intensity measurements does not allow a distinction to be made—in particular for low fluorescence intensity values—between a fluorescence intensity value which is low but reflects the presence of a low concentration of anti-HLA antibodies, and a low fluorescence intensity value which cannot be distinguished from the background noise of the measurement.

For the same reasons as set out above, such a preparation used as a positive control in the prior art does not allow the concentration of antibodies present in the liquid medium, especially in a serum collected from a transplant patient or a patient awaiting a transplant, to be determined precisely.

The invention aims to remedy these disadvantages by proposing a monoclonal chimeric immunoglobulin as a standardization and positive control and sensitivity reagent in the serological analysis of anti-HLA class I antibodies or anti-HLA class II antibodies, especially within the context of organ transplantation.

The invention aims to remedy the disadvantages discussed above by proposing a monoclonal chimeric immunoglobulin which is suitable for permitting reliable quantification of anti-HLA antibodies in the serum of a patient. Such a monoclonal chimeric immunoglobulin is suitable in particular for permitting the detection of the occurrence in a patient of antibodies directed against the antigens of the transplanted organ, even for a very low concentration of that antibody.

The invention aims to propose a monoclonal chimeric immunoglobulin which is suitable for the standardization of anti-HLA antibody detection methods. By allowing in particular the detection threshold to be defined precisely, the monoclonal chimeric immunoglobulin according to the invention allows the biologist to validate the antibody detection method.

The invention aims to propose a monoclonal chimeric immunoglobulin which is suitable for permitting the detection as early as possible of the occurrence of anti-HLA antibodies directed against the HLA antigens of the transplanted organ. Such early detection in particular allows humoral rejection to be discovered as quickly as possible and therefore enables the curative treatment for rejection of the transplanted organ to be prescribed quickly.

The invention aims to propose such a monoclonal chimeric immunoglobulin which allows methods for the detection of anti-HLA antibodies in a biological liquid to be standardized and calibrated.

The invention aims also to propose such a monoclonal chimeric immunoglobulin which is capable of permitting the accreditation of a standardized method for the screening, quantification and characterization of anti-HLA antibodies according to the new regulatory standards for accreditation of medical analysis laboratories. In France, medical analysis—especially immunology—laboratories must comply with COFRAC (French accreditation committee) standard 15189.

The invention aims also to propose a composition of at least one monoclonal chimeric immunoglobulin as quantitative calibration reagent in tests for screening anti-HLA antibodies, especially by immunofluorimetry.

The invention aims in particular to propose such a composition of at least one monoclonal chimeric immunoglobulin as quantification standard in tests for screening anti-HLA antibodies by “Luminex®” technology.

The invention aims also to propose such a composition of at least one chimeric immunoglobulin as quantitative standard in tests for screening anti-HLA antibodies by flow cytometry, by the ELISA technique or by complement-dependent microlymphocytotoxicity.

The invention therefore aims to propose such a monoclonal chimeric immunoglobulin and such a composition of at least one monoclonal chimeric immunoglobulin for use as quantitative standard instead of a complex mixture of antibodies obtained from the mixture of one or more serums of patients.

The invention aims also to propose such a monoclonal chimeric immunoglobulin which is available in a large quantity and the production of which is perfectly standardized in terms of anti-HLA antibody concentration and in terms of composition.

The invention aims in addition to propose a stable aqueous solution of such a monoclonal chimeric immunoglobulin, the concentration of monoclonal chimeric immunoglobulin of which is known perfectly, for the standardization of a method for the screening and/or quantification and/or characterization of anti-HLA antibodies in a liquid medium.

To that end, the invention relates to a process for determining the quantity of anti-HLA antibodies in a liquid medium susceptible to contain antibodies, the process comprising:
determining a calibration curve, said calibration curve being defined by a plurality of measured values of a parameter, each measured value (Vₙ) of the plurality of values corresponding to a determined quantity (Qₙ) of a monoclonal chimeric immunoglobulin according to the invention;
measuring a value (Vₓ) of the parameter corresponding to the liquid medium, and
deducing the quantity of anti-HLA antibody in the liquid medium from the calibration curve and from the measured value (Vₓ) of the parameter corresponding to the liquid medium;
   wherein the monoclonal chimeric immunoglobulin consists of:
two polypeptide heavy chains (H), of molecular weight from 40 kDa to 60 kDa, and
two polypeptide light chains (L), of molecular weight from 20 kDa to 30 kDa, wherein:
each heavy chain (H) comprises:
a heavy chain variable region (V_{H}) of a monoclonal antibody selected from the group consisting of monoclonal antibodies specific to monomorphic epitopes of HLA class I antigens and monoclonal antibodies specific to monomorphic epitopes of HLA class II antigens, and
a heavy chain constant region (C_{H}) of a human immunoglobulin chosen from the group formed of IgAs, IgGs and IgMs,
and wherein:
each light chain (L) comprises:
a light chain variable region (V_{L}) of a monoclonal antibody selected from the group consisting of monoclonal antibodies specific to monomorphic epitopes of HLA class I antigens and monoclonal antibodies specific to monomorphic epitopes of HLA class II antigens, and
a light chain constant region (C_{L}) of a human immunoglobulin selected from the group consisting of the kappa chains and the lambda chains.

According to the invention, the liquid medium is prepared from a biological fluid collected from a patient, especially from a serum from of a transplant patient or a patient awaiting a transplant.

The calibration curve is defined by the plurality of measured values of the parameter corresponding to various determined quantities of the monoclonal chimeric immunoglobulin according to the invention.

In a method according to the invention, the determination of the calibration curve comprises the steps of:
preparing a plurality of solutions (Sₙ), especially aqueous solutions, of a monoclonal chimeric immunoglobulin, each solution (Sₙ) comprising a determined quantity (Qₙ) of said monoclonal chimeric immunoglobulin and having a determined concentration value (Cₙ) of said monoclonal chimeric immunoglobulin, and then
placing a predetermined volume of each solution (Sₙ) in contact with a same determined quantity of at least one immobilized HLA antigen, and
measuring a value, named measured value (Vₙ), of a parameter, said measured value (Vₙ) being related to the quantity of the monoclonal chimeric immunoglobulin bound to the defined quantity of each immobilized HLA antigen,
forming pairs (Cₙ, Vₙ) of the defined concentration (Cₙ) and the measured value (Vₙ) representing the variation of the measured value (Vₙ) as a function of the defined concentration (Cₙ) of monoclonal chimeric immunoglobulin of each solution (Sₙ) of monoclonal chimeric immunoglobulin (dose/response data),
carrying out a statistical analysis—especially by sigmoid non-linear regression of the Boltzmann type—of the (Cₙ, Vₙ) pairs and determining the calibration curve by this statistical analysis.

In a process according to the invention, there is calculated a value, called a threshold value, of the parameter beyond which the concentration of monoclonal chimeric immunoglobulin is significantly greater than 0.

In a process for determining the quantity of anti-HLA antibodies in a liquid medium, the liquid medium is obtained from a biological fluid collected from a patient, the liquid medium having the quantity of anti-HLA antibodies to be determined.

A process according to the invention comprises the steps of:
placing the same predetermined volume of the liquid medium or of a dilution thereof in contact with the same defined quantity of at least one immobilized HLA antigen, and
measuring a (Vₓ) value associated with the volume of liquid medium
deducting the quantity of antibody in the liquid medium from the calibration curve and from the measured (Vₓ) value of the parameter corresponding to the concentration (Cₓ) of antibody in the liquid medium.

Throughout the text, the expressions “HLA class I antigen” and “HLA class II antigen” (HLA for “Human Leucocyte Antigen”) denote antigens which are human by nature.

The invention therefore consists also in proposing a process for the in vitro quantification of anti-HLA antibodies, wherein a calibration curve is produced from a plurality of solutions of at least one monoclonal chimeric immunoglobulin according to the invention, each solution of the plurality of solutions of said at least one monoclonal chimeric immunoglobulin having a known concentration of monoclonal chimeric immunoglobulin.

The inventors have in fact found that no solution is known in the prior art for permitting a reliable and reproducible quantitative evaluation of the concentration of anti-HLA class I and class II antibodies in a liquid medium.

Advantageously and according to the invention, the parameter is chosen from the group formed of fluorescence parameters, luminescence parameters and colorimetry parameters.

Advantageously and according to the invention, the defined concentration (Cₙ) of monoclonal chimeric immunoglobulin is not more than 10⁻⁴ g/ml, especially from 10⁻¹⁰ g/ml to 10⁻⁴ g/ml. Any concentration below 10⁻⁴ g/ml can be obtained by diluting a solution of high concentration, in particular of approximately 10⁻⁴ g/ml.

Advantageously, increasing progressive dilutions of the monoclonal chimeric immunoglobulin according to the invention are prepared. These solutions (Sₙ) of monoclonal chimeric immunoglobulin according to the invention of known concentrations are used to associate each concentration (Cₙ) of the solutions (Sₙ) of monoclonal chimeric immunoglobulin with a measured value (Vₙ) of a parameter chosen from the group formed of a fluorescence parameter, especially a fluorescence parameter measured by quantitative immunofluorimetry (Luminex® technique using beads coated with HLA antigens) or by the flow cytometry technique (carried out with lymphocytes), a luminescence parameter, especially a chemiluminescence parameter, and a colorimetry parameter.

Advantageously and according to the invention, in a process according to the invention, each monoclonal antibody specific to monomorphic epitopes of HLA class I antigens and each monoclonal antibody specific to monomorphic epitopes of HLA class II antigens is chosen from the group formed of the monoclonal antibodies of a vertebrate, in particular the monoclonal antibodies of a mammal, especially of a mouse, rat, rabbit, hamster and of a human, and the monoclonal antibodies of a non-mammalian vertebrate, especially of an amphibian, of a bird, in particular of a galliforme.

Advantageously and according to the invention, the parameter is chosen from the group formed of fluorescence parameters, luminescence parameters and colorimetry parameters.

Advantageously and according to the invention, the fluorescence parameter is a fluorescence intensity. Accordingly, each value (Vₙ) of the fluorescence parameter is a fluorescence intensity.

Advantageously and according to the invention, each measured value (Vₙ) of the parameter is measured by a technique chosen from the group formed of multiplex quantitative immunofluorimetry, flow cytometry, a method of immunoenzymatic assay on a solid substrate (ELISA), a competitive binding method and a complement-dependent microlymphocytotoxicity method.

Advantageously, in a first variant of a process according to the invention:
a) each immobilized HLA antigen being an HLA antigen immobilized on the surface of particles of a solid substrate in the divided state formed of particles,
b) the immobilized HLA antigens and each solution of monoclonal chimeric immunoglobulin directed against the HLA antigens of the solid substrate are brought into contact under conditions suitable for forming a stable bond between the HLA antigens of the solid substrate and the monoclonal chimeric immunoglobulin of each solution of monoclonal chimeric immunoglobulin, and then
c) the monoclonal chimeric immunoglobulins that are not bound to the HLA antigens of the solid substrate are removed by washing, and then
d) the monoclonal chimeric immunoglobulins that are bound to the HLA antigens of the solid substrate are brought into contact with a solution of a secondary antibody which is chosen from the group formed of fluorescent secondary antibodies, luminescent secondary antibodies and photoabsorbent secondary antibodies and which is directed against the monoclonal chimeric immunoglobulin, under conditions suitable for forming a stable bond between the monoclonal chimeric immunoglobulin and the secondary antibody, and then
e) the secondary antibody that is not bound to the monoclonal chimeric immunoglobulin is removed by washing, and then
f) at least one parameter of the secondary antibody that is bound to each particle of the solid substrate is measured, and there is assigned to that measurement a measured value (Vₙ) of said parameter chosen from the group formed of a fluorescence parameter, especially a fluorescence parameter measured by quantitative immunofluorimetry (Luminex® technique using beads coated with HLA antigens) or by the flow cytometry technique (carried out with lymphocytes), a luminescence parameter, especially a chemiluminescence parameter, and a colorimetry parameter, and then
g) the calibration curve is formed.

Advantageously, in the first variant of a process according to the invention:
h) there is derived from the calibration curve a fluorescence intensity threshold value indicating the presence of the anti-HLA antibody in a solution to be analysed.

Advantageously, in a second variant and according to the invention, each immobilized HLA antigen is an HLA antigen presented on the surface of at least one cell, especially a cell in in vitro culture.

Advantageously, in this second variant of a process according to the invention:
i) each immobilized HLA antigen being an HLA antigen presented on the surface of at least one cell,
j) the HLA antigens presented on the surface of at least one cell and each solution of monoclonal chimeric immunoglobulin are brought into contact under conditions suitable for forming a stable bond between the HLA antigens of the cell(s) and the monoclonal chimeric immunoglobulin of each solution of monoclonal chimeric immunoglobulin, and then
k) the monoclonal chimeric immunoglobulin that is not bound to the HLA antigens of the cell(s) is removed by washing, and then
l) the monoclonal chimeric immunoglobulin that is bound to the HLA antigens of the cell(s) is brought into contact with a solution of a secondary antibody directed against the monoclonal chimeric immunoglobulin, under conditions suitable for forming a stable bond between the monoclonal chimeric immunoglobulin and the secondary antibody, and then
m) the secondary antibody that is not bound to the monoclonal chimeric immunoglobulin is removed by washing, and then
n) at least one parameter of the secondary antibody that is bound to each cell is measured, and there is assigned to that measurement a measured value (Vₙ) of said parameter chosen from the group formed of a fluorescence parameter, especially a fluorescence parameter measured by quantitative immunofluorimetry (Luminex® technique using beads coated with HLA antigens) or by the flow cytometry technique (carried out with lymphocytes), a luminescence parameter, especially a chemiluminescence parameter, and a colorimetry parameter, and then
o) the calibration curve is formed.

Advantageously, in the second variant of a process according to the invention:
p) there is derived from the calibration curve a fluorescence intensity threshold value indicating the presence of the anti-HLA antibody in a solution to be analysed.

Advantageously and according to the invention, the solid substrate in the divided state is in the form of particles of substantially spherical shape and of a size suitable for permitting their analysis by flow fluorimetry.

Advantageously and according to the invention, the calibration curve is determined by non-linear regression from fluorescence intensity measurements.

Advantageously and according to the invention, the liquid medium is chosen from the group formed of biological fluids, especially a serum, collected from an individual.

Advantageously and according to the invention, the individual is a patient chosen from the group formed of patients awaiting a transplant and transplant patients.

Advantageously and according to the invention, the monoclonal antibody specific to monomorphic epitopes of HLA class I antigens is the W6/32 antibody.

Advantageously and according to the invention, the monoclonal antibody specific to monomorphic epitopes of HLA class II antigens is the F3.3 antibody.

The invention relates also to the use of a monoclonal chimeric immunoglobulin in a screening method, especially a method for the detection or a method for the identification or quantification of anti-HLA antibodies chosen from the group formed of multiplex quantitative immunofluorimetry methods, flow cytometry methods, methods of immunoenzymatic assay on a solid substrate (ELISA) and complement-dependent microlymphocytotoxicity methods.

The invention relates in particular to the use of a monoclonal chimeric immunoglobulin as a standardization and positive control and sensitivity reagent in a method for the screening or quantification of anti-HLA antibodies chosen from the group formed of multiplex quantitative immunofluorimetry methods, flow cytometry methods, methods of immunoenzymatic assay on a solid substrate, and complement-dependent microlymphocytotoxicity methods, wherein the monoclonal chimeric immunoglobulin is formed of:
two polypeptide heavy chains (H), of molecular weight from 40 kDa to 60 kDa, and
two polypeptide light chains (L), of molecular weight from 20 kDa to 30 kDa, wherein:
each heavy chain (H) comprises:
a heavy chain variable region (V_{H}) of a monoclonal antibody selected from the group consisting of monoclonal antibodies specific to monomorphic epitopes of HLA class I antigens and monoclonal antibodies specific to monomorphic epitopes of HLA class II antigens, and
a heavy chain constant region (C_{H}) of a human immunoglobulin selected from the group consisting of IgAs, IgGs and IgMs,
and wherein:
each light chain (L) comprises:
a light chain variable region (V_{L}) of a monoclonal antibody selected from the group consisting of monoclonal antibodies specific to monomorphic epitopes of HLA class I antigens and monoclonal antibodies specific to monomorphic epitopes of HLA class II antigens, and
a light chain constant region (C_{L}) of a human immunoglobulin selected from the group consisting of the kappa chains and the lambda chains.

There is therefore used a monoclonal chimeric immunoglobulin according to the invention in which the heavy chain constant parts (C_{H}) and the light chain constant parts (C_{L}) are constituted by the constant parts of a human IgA in competition tests. Such class IgA monoclonal chimeric immunoglobulins according to the invention are suitable for inhibiting at least partially the fixing of anti-HLA class IgG (or IgM) polyclonal antibodies present in the serum of the patients. The inhibition of the fixing of the IgGs of the serum by the class IgA monoclonal chimeric immunoglobulins according to the invention makes it possible to demonstrate the specificity of the fixing of the IgGs to the immunoadsorbent substrates used in the tests for screening anti-HLA antibodies by multiplex quantitative immunofluorimetry with reading in flow on a “Luminex®” apparatus, by flow cytometry, by ELISA techniques, and by complement-dependent microlymphocytotoxicity.

In particular, the class IgA monoclonal chimeric immunoglobulins make it possible to differentiate a specific signal corresponding to the actual presence of anti-HLA IgG in the serum from a non-specific signal corresponding to the adsorption of the IgGs on the substrate having HLA antigens. This competition of the class IgA monoclonal chimeric immunoglobulins according to the invention for the detection of anti-HLA IgG has been demonstrated by quantitative immunofluorimetry on Luminex®.

The class IgG and class IgM monoclonal chimeric immunoglobulins according to the invention can be used as positive controls and sensitivity controls for direct compatibility testing (cross-match) between a recipient and an organ donor carried out just before the organ transplant by any suitable technique (complement-dependent microlymphocytotoxicity or flow cytometry) and in any technique for detection of class IgG or IgM antibodies on an immunoabsorbent substrate (ELISA or quantitative immunofluorimetry on polystyrene beads).

The invention relates also to a monoclonal chimeric immunoglobulin specific to HLA class I antigens, formed of:
two polypeptide heavy chains (H), of molecular weight from 40 kDa to 60 kDa, and
two polypeptide light chains (L), of molecular weight from 20 kDa to 30 kDa, wherein:
each heavy chain (H) comprises:
a heavy chain variable region (V_{H}) of a monoclonal antibody selected from the group consisting of monoclonal antibodies specific to monomorphic epitopes of HLA class I antigens, and
a heavy chain constant region (C_{H}) of a human immunoglobulin selected from the group consisting of IgAs, IgGs and IgMs,
and wherein:
each light chain (L) comprises:
a light chain variable region (V_{L}) of a monoclonal antibody selected from the group consisting of monoclonal antibodies specific to monomorphic epitopes of HLA class I antigens, and
a light chain constant region (C_{L}) of a human immunoglobulin selected from the group consisting of the kappa chains and the lambda chains.

Advantageously, a monoclonal antibody specific to a monomorphic epitope of HLA class I antigens is formed to be able to recognize an epitope common to all HLA class I antigens.

Advantageously and according to the invention, the monoclonal chimeric immunoglobulin specific to HLA class I antigens is selected from the group consisting of:
monoclonal chimeric immunoglobulins comprising at least one light chain of sequence SEQ ID_NO 1, and
monoclonal chimeric immunoglobulins comprising at least one heavy chain selected from the group consisting of heavy chain of sequence SEQ ID_NO 2, heavy chain of sequence SEQ ID_NO 3 and heavy chain of sequence SEQ ID_NO 4.

The invention relates also to a monoclonal chimeric immunoglobulin specific to HLA class II antigens, formed of:
two polypeptide heavy chains (H), of molecular weight from 40 kDa to 60 kDa, and
two polypeptide light chains (L), of molecular weight from 20 kDa to 30 kDa, wherein it is selected from the group consisting of:
monoclonal chimeric immunoglobulins comprising at least one light chain of sequence SEQ ID_NO 5, and
monoclonal chimeric immunoglobulins comprising at least one heavy chain selected from the group consisting of heavy chain of sequence SEQ ID_NO 6, heavy chain of sequence SEQ ID_NO 7 and heavy chain of sequence SEQ ID_NO 8.

Advantageously, a monoclonal antibody specific to a monomorphic epitope of anti-HLA class II antigens is formed to be able to recognize an epitope common to substantially all HLA class II antigens.

Advantageously, a monoclonal chimeric immunoglobulin specific to HLA class I or class II antigens according to the invention denotes an immunoglobulin in which:
the heavy chains and the light chains are human by nature in their constant parts. In particular, the heavy chain constant parts are chosen from the group formed of the heavy chain constant parts of an IgA, the heavy chain constant parts of an IgG and the heavy chain constant parts of an IgM, and the light chain constant parts are chosen from the group formed of the kappa chains and the lambda chains, and
the light chain and heavy chain variable parts are chosen from the group formed of monoclonal antibodies specific to monomorphic epitopes of HLA class I antigens and monoclonal antibodies specific to monomorphic epitopes of HLA class II antigens. In such a monoclonal chimeric immunoglobulin, the heavy chain constant parts (C_{H}) of the human antibody and the light chain constant parts (C_{L}) of the human antibody together represent approximately 60% by mass of the monoclonal chimeric immunoglobulin. In addition, such a monoclonal chimeric immunoglobulin is a monoclonal antibody, that is to say specific to a single monomorphic epitope.

Advantageously, each monoclonal antibody is chosen from the group formed of the monoclonal antibodies of a vertebrate organism, especially a non-human vertebrate organism, specific to monomorphic epitopes of HLA class I antigens and the monoclonal antibodies of a vertebrate organism, especially a non-human vertebrate organism, specific to monomorphic epitopes of HLA class II antigens.

Throughout the text, “non-human vertebrate organism” is understood as being a superior organized living being with the exception of a human. Such a vertebrate organism is in particular provided with an immune system. Such a vertebrate organism is in particular chosen from the group formed of non-human mammals, especially mice, rats, rabbits and hamsters, amphibians and birds, especially galliformes. Such non-human vertebrate organisms are in particular laboratory animals. However, such a monoclonal antibody can be chosen from the group formed of human monoclonal antibodies.

Advantageously and according to the invention, each monoclonal antibody specific to monomorphic epitopes of HLA class I antigens and each monoclonal antibody specific to monomorphic epitopes of HLA class II antigens is chosen from the group formed of the monoclonal antibodies of a vertebrate, in particular the monoclonal antibodies of a mammal, especially of a mouse, rat, rabbit, hamster and of a human, and the monoclonal antibodies of a non-mammalian vertebrate, especially of an amphibian, of a bird, in particular of a galliforme.

Advantageously and according to the invention, the monoclonal antibody specific to monomorphic epitopes of HLA class I antigens is the W6/32 antibody.

Advantageously and according to the invention, the monoclonal antibody specific to monomorphic epitopes of HLA class II antigens is the F3.3 antibody.

The invention relates in addition to a stable solution of at least one monoclonal chimeric immunoglobulin according to the invention in an aqueous composition.

The invention relates also to such an aqueous solution of at least one monoclonal chimeric immunoglobulin according to the invention, said aqueous solution having a predetermined concentration of said monoclonal chimeric immunoglobulin.

The invention relates also to such a monoclonal chimeric immunoglobulin which is suitable for permitting a determination of the quantity of anti-HLA antibody in a liquid medium containing antibodies.

The invention extends in addition to a kit for the in vitro quantification of anti-HLA antibodies in a liquid medium, said kit comprising a predetermined quantity of at least one monoclonal chimeric immunoglobulin and instructions for carrying out a process according to the invention.

Accordingly, the invention relates to a kit for the in vitro quantification of anti-HLA antibodies in a liquid medium.

Accordingly, the invention relates to a kit for the in vitro quantification of anti-HLA antibodies in a liquid medium, said kit comprising a predetermined quantity of at least one monoclonal chimeric immunoglobulin comprising:
two polypeptide heavy chains (H), of molecular weight from 40 kDa to 60 kDa, and
two polypeptide light chains (L), of molecular weight from 20 kDa to 30 kDa, wherein:
each heavy chain (H) comprises:
a heavy chain variable region (V_{H}) of a monoclonal antibody selected from the group consisting of monoclonal antibodies specific to monomorphic epitopes of HLA class I antigens and monoclonal antibodies specific to monomorphic epitopes of HLA class II antigens, and
a heavy chain constant region (C_{H}) of a human immunoglobulin selected from the group consisting of IgAs, IgGs and IgMs,
and wherein:
each light chain (L) comprises:
a light chain variable region (V_{L}) of a monoclonal antibody selected from the group consisting of monoclonal antibodies specific to monomorphic epitopes of HLA class I antigens and monoclonal antibodies specific to monomorphic epitopes of HLA class II antigens, and
a light chain constant region (C_{L}) of a human immunoglobulin selected from the group consisting of the kappa chains and the lambda chains,
said kit also comprising instructions for carrying out a process according to the invention.

The invention relates also to a process for determining the quantity of anti-HLA antibodies in a liquid medium containing antibodies, a monoclonal chimeric immunoglobulin, its use, and a kit for that determination, characterized in combination by all or some of the features mentioned hereinabove or hereinbelow.

Other objects, features and advantages of the invention will become apparent upon reading the following description, which makes reference to the accompanying figures showing preferred embodiments of the invention, given solely by way of non-limiting examples, and in which:

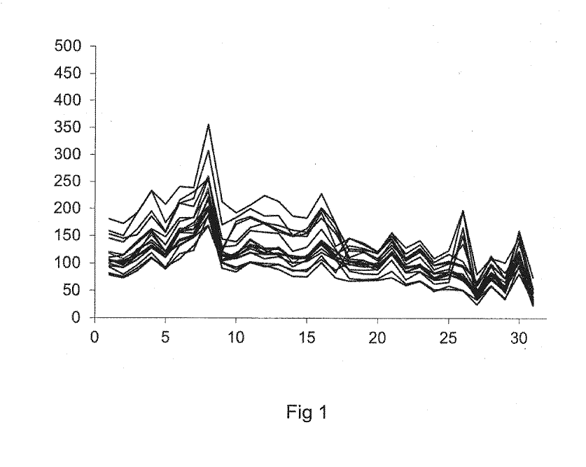

FIG. 1 is a graphical representation of the variation over time of the fluorescence intensity of 17 types of beads treated according to a negative control;

FIG. 2 is a graphical representation of the variation of the fluorescence intensity of 12 types of beads carrying HLA class I antigens of a positive control according to the prior art as a function of time;

FIG. 3 is a graphical representation of the variation of the fluorescence intensity of 5 types of HLA class II beads of a positive control according to the prior art as a function of time;

FIG. 4A and FIG. 4B are comparative representations in histogram form of the fluorescence intensity of a positive control according to the prior art and a positive control according to the invention, in which:
FIG. 4A is a representation in histogram form of the fluorescence intensity measured on 12 types of HLA class I beads treated with a positive control according to the prior art (hatched histogram) and with a positive control according to the invention (solid histogram);
FIG. 4B is a representation in histogram form of the fluorescence intensity measured on 5 types of HLA class II beads treated with a positive control according to the prior art (hatched histogram) and with a positive control according to the invention (solid histogram);

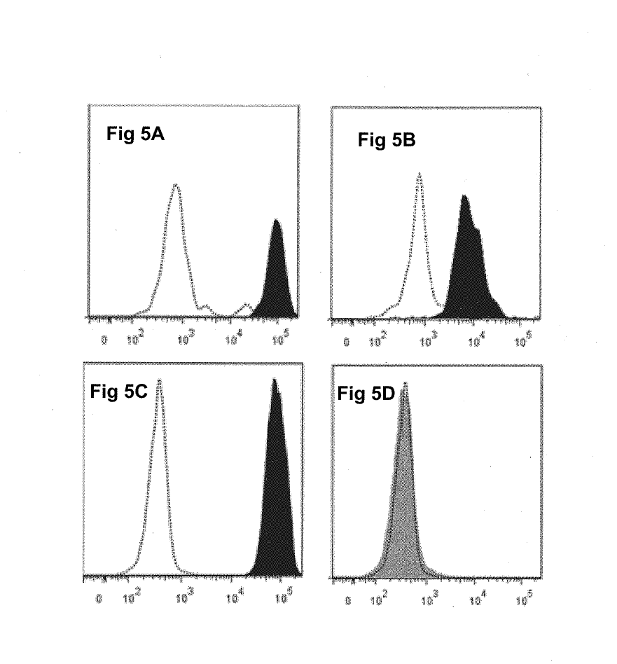

FIG. 5A, FIG. 5B, FIG. 5C, and FIG. 5D depict analysis by flow cytometry of the fixing of class I and class II chimeric monoclonal immunoglobulins according to the invention to T lymphocytes and B lymphocytes; the results show that the Hu-IgG1 K [W6/32] antibody fixes itself to the B lymphocytes (FIG. 5A) and to the T lymphocytes (FIG. 5C), whereas the Hu-IgG1 K [F3.3] antibody fixes itself to the B lymphocytes (FIG. 5B) and not to the T lymphocytes (FIG. 5D);

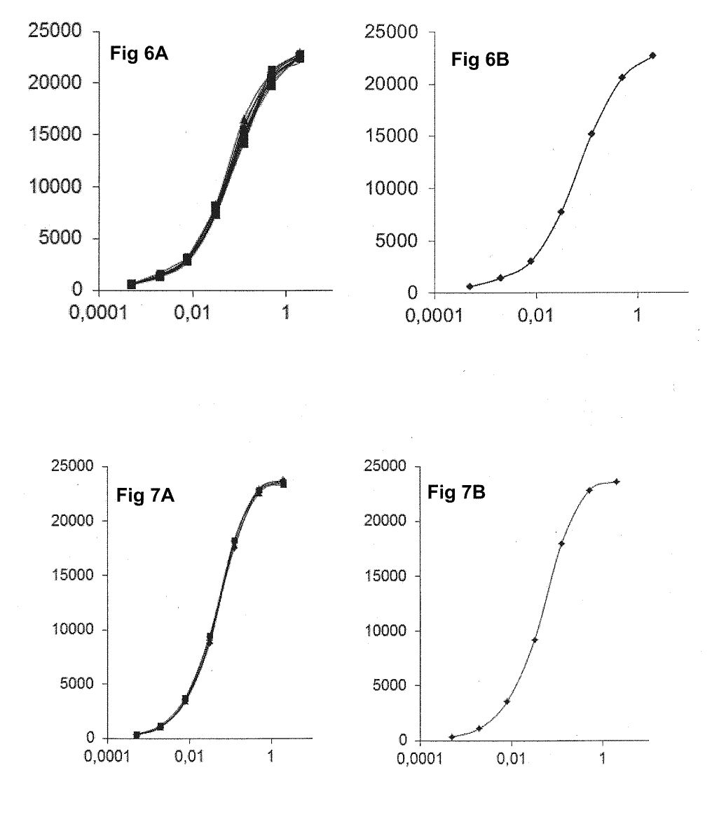

FIG. 6A is a graphical representation of 12 superposed dose/response curves corresponding to 12 types of HLA class I beads treated with a positive control according to the invention. The concentration of monoclonal chimeric immunoglobulin according to the invention is expressed in μg/ml;

FIG. 6B is a graphical representation of the average dose/response curve of 12 types of HLA class I beads treated with a positive control according to the invention corresponding to FIG. 6A. The concentration of monoclonal chimeric immunoglobulin according to the invention is expressed in μg/ml;

FIG. 7A is a graphical representation of 5 superposed dose/response curves corresponding to 5 types of HLA class II beads treated with a positive control according to the invention. The concentration of monoclonal chimeric immunoglobulin according to the invention is expressed in μg/ml;

FIG. 7B is a graphical representation of the average dose/response curve of the 5 types of HLA class II beads treated with a positive control according to the invention corresponding to FIG. 7A. The concentration of monoclonal chimeric immunoglobulin according to the invention is expressed in μg/ml;

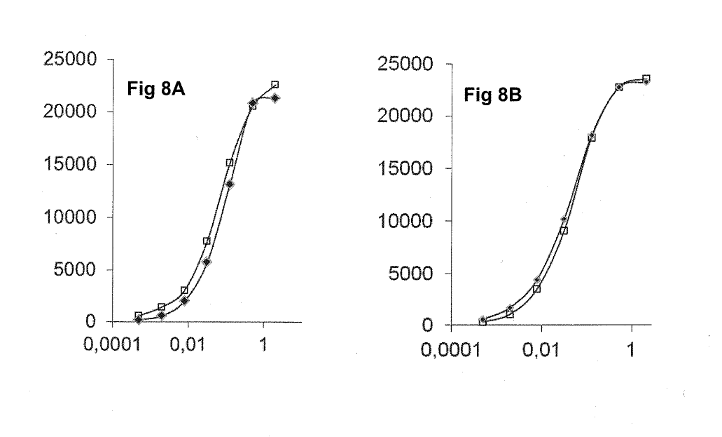

FIG. 8A is a comparative graphical representation of the dose/response curve of an anti-HLA class I monoclonal chimeric immunoglobulin according to the invention stored in a saline buffer (white square) and stored in a solution of human albumin at 60 g/l (black lozenge);

FIG. 8B is a comparative graphical representation of the dose/response curve of an anti-HLA class II monoclonal chimeric immunoglobulin according to the invention stored in a saline buffer (white square) and stored in a solution of human albumin at 60 g/l (black lozenge);

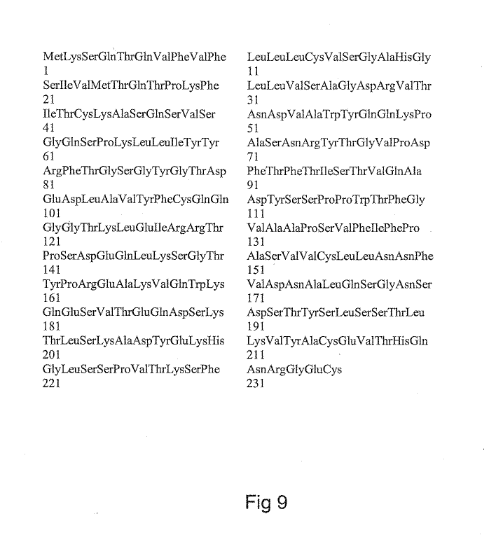

FIG. 9 is a representation of the peptide sequence of the light chain of the anti-HLA class I monoclonal chimeric immunoglobulin (Hu-IgG1 K [W6/32]) and corresponds to sequence SEQ ID_NO 1;

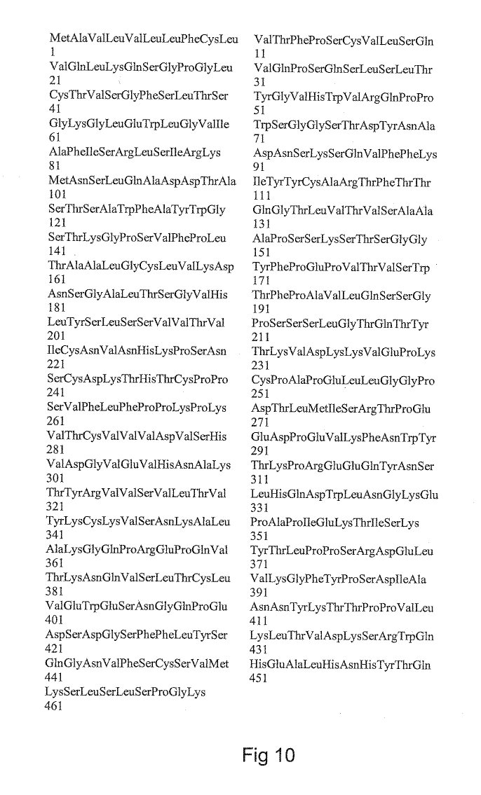

FIG. 10 is a representation of the peptide sequence of the heavy chain of the monoclonal chimeric immunoglobulin Hu-IgG1 K [W6/32] and corresponds to sequence SEQ ID_NO 2;

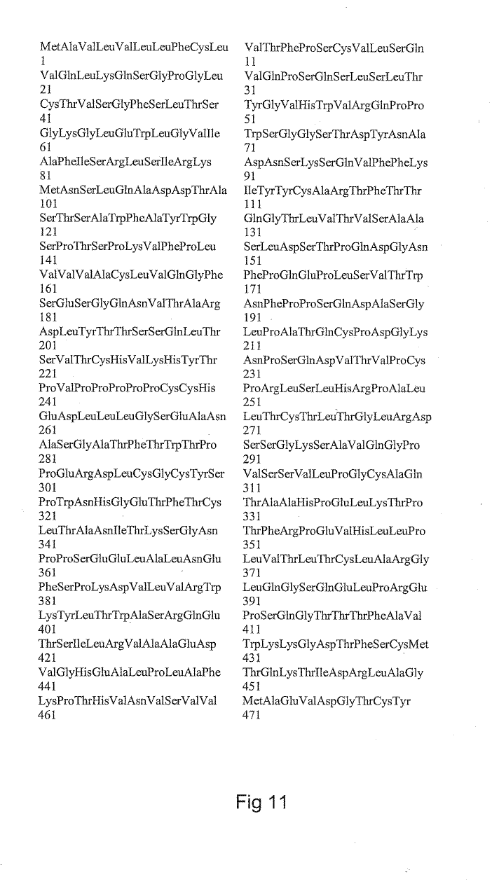

FIG. 11 is a representation of the peptide sequence of the heavy chain of the monoclonal chimeric immunoglobulin Hu-IgA2 K [W6/32] and corresponds to sequence SEQ ID_NO 3;

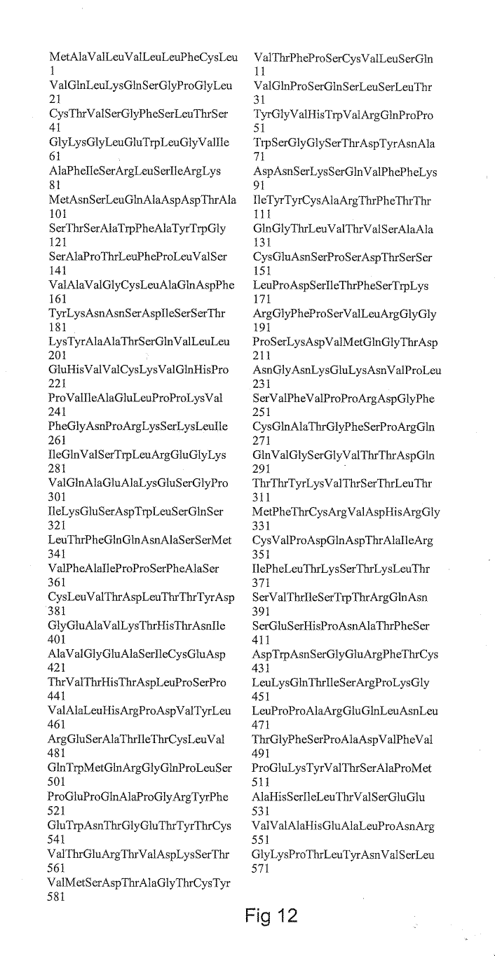

FIG. 12 is a representation of the peptide sequence of the heavy chain of the monoclonal chimeric immunoglobulin Hu-IgM K [W6/32] and corresponds to sequence SEQ ID_NO 4;

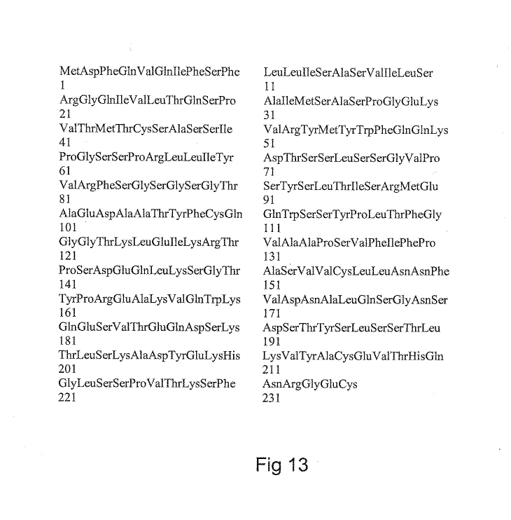

FIG. 13 is a representation of the peptide sequence of the light chain of the monoclonal chimeric immunoglobulin Hu-IgG1 K [F3.3] and corresponds to sequence SEQ ID_NO 5;

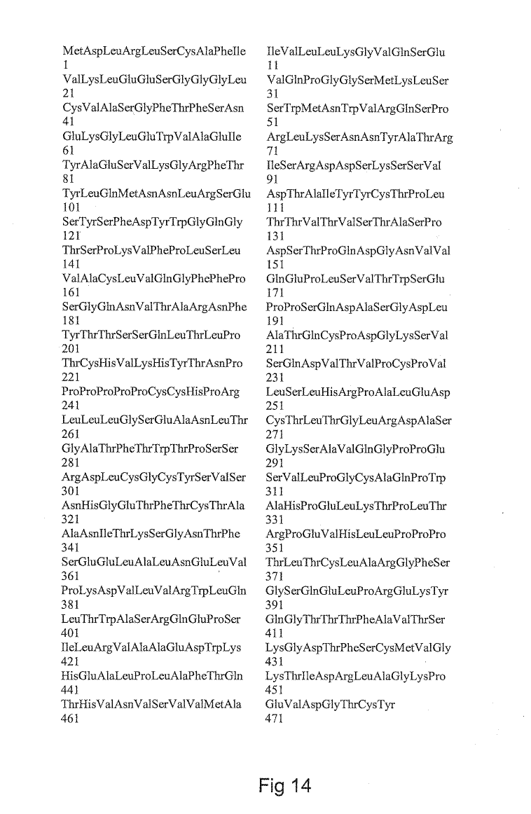

FIG. 14 is a representation of the peptide sequence of the heavy chain of the monoclonal chimeric immunoglobulin Hu-IgA2 K [F3.3] and corresponds to sequence SEQ ID_NO 6;

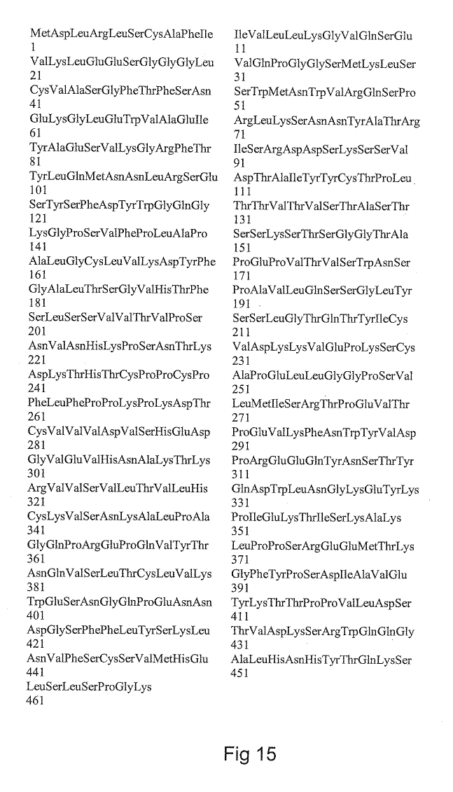

FIG. 15 is a representation of the peptide sequence of the heavy chain of the monoclonal chimeric immunoglobulin Hu-IgG1 K [F3.3] and corresponds to sequence SEQ ID_NO 7;

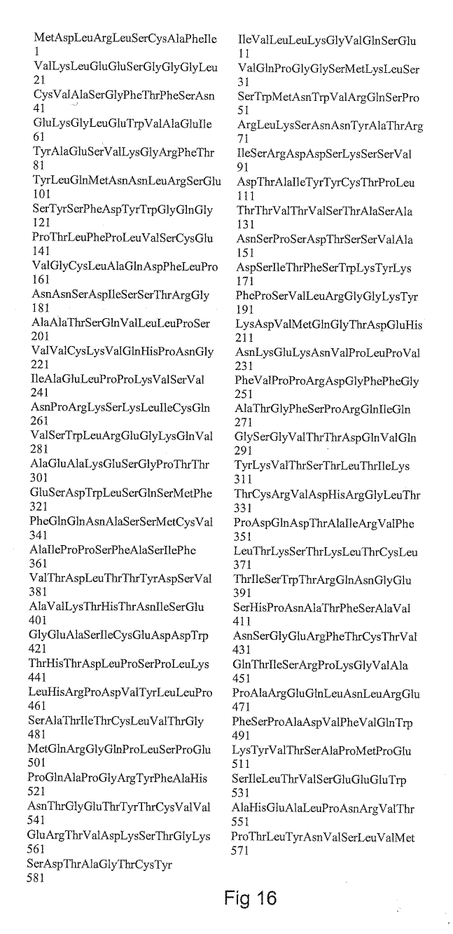

FIG. 16 is a representation of the peptide sequence of the heavy chain of the monoclonal chimeric immunoglobulin Hu-IgM K [F3.3] and corresponds to sequence SEQ ID_NO 8.

### EXAMPLE 1—PRODUCTION OF A IgG/ANTI-HLA CLASS I MONOCLONAL CHIMERIC IMMUNOGLOBULIN (HU-IgG1 K [W6/32]) ACCORDING TO THE INVENTION

The W6/32 antibody was described for the first time in the publication of Barnstable et al. in 1978 (Barnstable C J, Bodmer W F, Brown G, Galfre G, Milstein C, Williams A F, Ziegler A., 1978, Celle, 14(1), 9-20. Production of monoclonal antibodies to group A erythrocytes, HLA and other human cell surface antigens—new tools for genetic analysis). This antibody is secreted by a mouse hybridoma coming from the fusion of cells of a mouse myeloma line (P3-NSI/1Ag4-1 line which does not secrete mouse immunoglobulin) with the lymphocytes of a mouse immunized against human cells. It has been shown that this antibody reacts with a public epitope found on the HLA class I molecules (HLA-A, HLA-C). The recognized epitope is a conformational epitope which depends on the association between the class I alpha heavy chain and beta-2 microglobulin. The epitope requires the presence of an arginine at position 3 of the beta-2 microglobulin and a lysine at position 121 of the alpha chain (Ladasky J J, Shum B P, Canavez F, Seuánez H N, Parham P., (1999), Immunogenetics, 49(4), 312-320. Residue 3 of beta2-microglobulin affects binding of class I MHC molecules by the W6/32 antibody).

In a first step, the transcripts coding for the heavy chain and the light chain of the W6/32 antibody were cloned and sequenced.

The DNA copy of the mRNAs (cDNAs) of the heavy chain was amplified by PCR with the aid of two primers, one specific to the region coding for the leader peptide, the other targeting the 5′ part which codes for the CH1 domain of the constant part.

For the amplification of the cDNA of the light chain, a primer targeting the exon of the leader peptide and a primer targeting the 5′ part of the C kappa domain were used. The cDNA fragments of the two chains were cloned in E. coli. The cloned fragments were sequenced. Alignment of the sequences allowed the consensus of the cDNAs of the heavy chain and of the light chain to be established with an identity threshold of 98%. The regions coding for the variable parts were determined by comparison with the sequences present in the IMGT® (“International Immunogenetics Information System”) data bank.

Sequences coding for a light chain or heavy chain leader peptide, as appropriate, were added at 5′, and the sequences at 3′ were modified so as to create a BsiWI restriction site for the DNA of the light chain and a NheI restriction site for the heavy chain. These restriction sites are suitable for permitting an insertion of these nucleic sequences into the cloning vectors pFUSE-CLIg and pFUSE-CHIg (InvivoGen, Toulouse, France). The sequences so defined were synthesized and then cloned in expression vectors comprising either the part coding for the constant domain of the human kappa chain (allotype Km 01; Genbank accession number: J00241) or the constant part of the human IgG1s. The restriction site of the expression vector permits the insertion of the cDNAs of the variable parts in phase with the regions coding for the constant parts of the immunoglobulin chains.

Two expression vectors, one coding for a chimeric light chain associating the variable part of the light chain of the W6/32 antibody and the human C kappa domain (VL [W6/32]-human C kappa chain), the other associating the variable part of the heavy chain of the W6/32 antibody and the human C gamma 1 domain (VL [W6/32]-human C gamma 1) were thus obtained.

These two vectors were introduced by transfection into CHO cells (Chinese Hamster Ovary cells). To that end, the cells were first transfected by the vector coding for the light chain and then by the vector coding for the heavy chain. The expression vectors comprise cytotoxic drug resistance factors which allow the double-transfected cells to be selected effectively. After the selection period, the double-transfected cells were cloned by limiting dilution and the presence of the human IgG1 kappa was revealed in the supernatant of the clones by a sandwich ELISA technique. The producer clones so revealed were subjected to a plurality of cloning cycles by limiting dilution in such a manner as to recruit the most effective clones in the secretion of the chimeric IgG1 kappa, denoted Hu-IgG1 K [W6/32] hereinbelow.

The Hu-IgG1 K [W6/32] secreted in the culture supernatant of the transfected CHO cells were purified by capture elution on staphylococcal protein A bound to Sepharose® beads. The IgG1 kappa were eluted at acid pH in a glycine buffer at pH 2. The eluate was buffered extemporaneously with an aqueous solution of disodium phosphate at a concentration of 750 mM. The solutions of Hu-IgG1 K [W6/32] are stored at 4° C. or at −80° C.

The peptide sequence of the light chain of the monoclonal chimeric immunoglobulin (IgG1 anti-HLA class I) Hu-IgG1 K [W6/32] is shown in FIG. 9 and corresponds to sequence SEQ ID_NO 1. The peptide sequence of the heavy chain of the monoclonal chimeric immunoglobulin (IgG1 anti-HLA class I) Hu-IgG1 K [W6/32] is shown in FIG. 10 and corresponds to sequence SEQ ID_NO 2.

Verification of the specificity of the binding of the monoclonal chimeric immunoglobulin Hu-IgG1 K [W6/32] according to the invention was carried out on T lymphocytes or on B lymphocytes separated on a density gradient, starting from human blood collected using an EDTA tube. The cells were labelled with anti-CD3, anti-CD19 and anti-CD45 antibodies. The T lymphocytes (CD3+) and B lymphocytes (CD19+) are defined among the population of the CD45+ lymphocytes.

The monoclonal chimeric immunoglobulin Hu-IgG1 K [W6/32] is incubated in the presence of human mononuclear cells (T lymphocytes or B lymphocytes) of the peripheral blood, and then said human mononuclear cells are washed three times in succession followed by washing three times in phosphate buffered saline (PBS). The fixing of the monoclonal chimeric immunoglobulins fixed to the human mononuclear cells is revealed by means of goat anti-human Fcγ antibodies. The results are presented in FIG. 5A, FIG. 5B, FIG. 5C, and FIG. 5D and show that the Hu-IgG1 K [W6/32] antibody fixes itself to the B lymphocytes (FIG. 5A) and to the T lymphocytes (FIG. 5C), whereas the Hu-IgG1 K [F3.3] antibody fixes itself to the B lymphocytes (FIG. 5B) and not to the T lymphocytes (FIG. 5D).

### EXAMPLE 2—SPECIFICITY OF THE MONOCLONAL CHIMERIC IMMUNOGLOBULINS HU-IgG1 K [W6/32]

The specificity of the monoclonal chimeric immunoglobulins Hu-IgG1 K [W6/32] was then determined by a technique of multiplex quantitative fluorimetry using commercial kits distributed by One Lambda®. This determination is based on an indirect immunofluorescence reaction and uses latex beads coated with HLA antigen of different groups. The antibodies capable of recognizing the HLA antigens present on the latex beads are revealed by anti-human IgG antibodies coupled to phycoerythrin. The fluorescence is quantified on each bead by flow cytometry in a Luminex® apparatus. Specific fluorescent labelling of each type of beads allows several varieties of beads (recognizable by their fluorescence) to be used, each bead being coated with a given mixture of HLA antigens. This makes it possible to show that all the class I beads are recognized with substantially the same intensity (FIG. 4A, class I beads). This allows us to conclude that the monoclonal chimeric immunoglobulin Hu-IgG1 K [W6/32] recognizes a public epitope present on all the HLA class I molecules.

Competition experiments have made it possible to show that the W6/32 mouse monoclonal antibody secreted by the mouse hybridoma inhibits the fixing of the monoclonal chimeric immunoglobulin Hu-IgG1 K [W6/32].

### EXAMPLE 3—PRODUCTION OF ANTI-HLA CLASS I CHIMERIC MONOCLONAL ANTIBODIES OF ISOTYPE IgA2 AND IgM (HU-IgA2 K [W6/32] AND HU-IgM K [W6/32]) ACCORDING TO THE INVENTION

The W6/32 heavy chain variable part (VH[W6/32]) was cloned in two other expression vectors; the first permits the production of chimeric mu chains (variable part of W6/32 and constant part of the human mu heavy chain (Genbank accession number: AY510104.1), the other permits the production of chimeric alpha 2 chains (variable part of W6/32 and constant part of the human alpha 2 heavy chain, allotype A2m(1) (Genbank accession number: J00221). These two vectors were used to transfect cells of the line CHO previously transfected by the vector permitting expression of the human chimeric light chain VL[W6/32]-K. We thus isolated a clone of CHO cells secreting large quantities of monoclonal chimeric immunoglobulin Hu-IgA2 K [W6/32] and another clone secreting a monoclonal chimeric immunoglobulin Hu-IgM K [W6/32]. The chimeric IgAs were purified on an agarose substrate coupled to peptide M (InvivoGen, Toulouse, France) and the chimeric IgMs were purified on an agarose substrate coupled to protein L (InvivoGen, Toulouse, France) according to the supplier's recommendations.

The peptide sequence of the heavy chain of the monoclonal chimeric immunoglobulin (IgA2 anti-HLA class I) Hu-IgA2 K [W6/32] is shown in FIG. 11 and corresponds to sequence SEQ ID_NO 3.

The peptide sequence of the heavy chain of the monoclonal chimeric immunoglobulin (IgM anti-HLA class 1) is shown in FIG. 12 and corresponds to sequence SEQ ID_NO 4.

The reactivity of the monoclonal chimeric immunoglobulins Hu-IgA2 K [W6/32] and Hu-IgM K [W6/32] was verified by the Luminex® technique with Labscreen Mixed® kits (One Lambda®) and, as fluorescent secondary antibodies, goat anti-human IgA or anti-human IgM antibodies coupled to phycoerythrin.

### EXAMPLE 4—PRODUCTION OF AN ANTI-HLA CLASS II MONOCLONAL ANTIBODY (HU-IgG1 K [F3.3]) ACCORDING TO THE INVENTION

In terms of its principle, the process for producing an anti-HLA class II monoclonal antibody is comparable to the process for producing an anti-HLA class I monoclonal antibody (W6/32).

The F3.3 antibody (Elsasser, D., Valerius, T., Repp, R., Weiner, G. J., Deo, Y., Kalden, J. R., van de Winkel, J. G., Stevenson, G. T., Glennie, M. J. and Gramatzki, M., (1996), Blood, 87(9), 3803-3812. HLA class II as potential target antigen on malignant B cells for therapy with bispecific antibodies in combination with granulocyte colony-stimulating factor) is the product of a single mouse hybridoma clone. The F3.3 antibody recognizes at least one public epitope present on the surface of all human cells that express the HLA class II molecules. In particular, the F3.3 antibody recognizes all the DR antigens, all the DP antigens and all the antigens of group DQ2. The complete sequences coding for the variable parts of the F3.3 antibody are accessible on GenBank® (accession numbers: AY058910 [VL] for the light chain and AY058911 [VH] for the heavy chain).

The sequences of the variable parts were synthesized and cloned in phase in the cloning vectors pFUSE-CHIg (InvivoGen, Toulouse, France) for the heavy chains and pFUSE-CLIg (InvivoGen, Toulouse, France) for the light chains. The vector (pFUSE-CHIg) permits the production of the chimeric heavy chain VH[F3.3]-human C gamma 1 and the vector (pFUSE-CLIg) permits the production of the light chain VL[F3.3]-human C kappa. These two expression vectors are used to transfect cells of the line CHO as described in Example 1.

In addition, the variable part VH[F3.3] was cloned in expression vectors permitting the production of chimeric heavy chains VH[F3.3]-human C mu and VH[F3.3]-human C alpha 2.

The peptide sequence of the light chain of the anti-HLA class II monoclonal chimeric immunoglobulin (Hu-IgG1 K [F3.3]) is shown in FIG. 13 and corresponds to sequence SEQ ID_NO 5.

The peptide sequence of the heavy chain of the anti-HLA class II monoclonal chimeric immunoglobulin (Hu-IgA2 K [F3.3]) is shown in FIG. 14 and corresponds to sequence SEQ ID_NO 6.

The peptide sequence of the heavy chain of the anti-HLA class II monoclonal chimeric immunoglobulin (Hu-IgG1 K [F3.3]) is shown in FIG. 15 and corresponds to sequence SEQ ID_NO 7.

The peptide sequence of the heavy chain of the anti-HLA class II monoclonal chimeric immunoglobulin (Hu-IgM K [F3.3]) is shown in FIG. 16 and corresponds to sequence SEQ ID_NO 8.

The monoclonal chimeric immunoglobulins Hu-IgG1 K [F3.3], Hu-IgA2 K [F3.3] and Hu-IgM K [F3.3] directed against the HLA class II antigens were produced by CHO cells transfected by the appropriate vectors. The chimeric IgG1s were purified on protein A Sepharose. The chimeric IgAs were purified on peptide M-agarose (InvivoGen, Toulouse, France). The chimeric IgMs were purified on protein L-agarose (InvivoGen, Toulouse, France).

The specificity of the binding of the monoclonal chimeric immunoglobulin Hu-IgG1 K [F3.3] according to the invention is verified by a technique of indirect immunofluorescence by flow cytometry on mononuclear human cells as described in Example 1. The results are presented in FIG. 5A, FIG. 5B, FIG. 5C, and

FIG. 5D and show that the antibody Hu-IgG1 K [F3.3] according to the invention reacts strongly against the human cells, especially with the B lymphocytes (FIG. 5B) and with the T lymphocytes (FIG. 5D).

In addition, the specificity of the anti-HLA class II monoclonal chimeric immunoglobulins according to the invention is studied by the technique of multiplex quantitative immunofluorimetry on Luminex® with the aid of Labscreen Mixed® and Labscreen single Antigen® kits (One Lambda®).

The antibody Hu-IgG1 K [F3.3] fixed itself to all the beads carrying the HLA class II antigens of the Labscreen Mixed® kit and to all the beads carrying the HLA-DR, HLA-DP and HLA-DQ2 antigens of the Labscreen single antigen class II® kit.

It has been shown, by competition experiments, that the monoclonal chimeric immunoglobulins Hu-IgG1 K [F3.3] and Hu-IgA2 K [F3.3] recognize the same epitope. The reactivity of the monoclonal chimeric immunoglobulin Hu-IgA2 K [F3.3] was studied by the Luminex® technique with the Labscreen Mixed® kits (One-Lambda®) using goat anti-human IgA antibodies coupled to phycoerythrin to reveal the fixing of the monoclonal chimeric immunoglobulins Hu-IgA2 K [F3.3]. The reactivity of the monoclonal chimeric immunoglobulin Hu-IgA2 K [F3.3] is found to be identical with that of the monoclonal chimeric immunoglobulin Hu-IgG1 K [F3.3].

### EXAMPLE 5—PROCESS FOR THE IN VITRO QUANTIFICATION OF ANTI-HLA ANTIBODIES

In a process for the in vitro quantification of anti-HLA antibodies in a liquid medium containing antibodies by immunofluorescence according to the invention, there is used, by way of non-limiting example, a “Labscreen Mixed®” laboratory kit (LSM12, One Lambda® Inc., USA) comprising:
polystyrene beads covalently bonded to purified HLA class I antigens (HLA-A, HLA-B and HLA-C), and, in admixture,
polystyrene beads covalently bonded to purified HLA class II antigens (HLA-DR, HLA-DQ and HLA-DP),
polystyrene beads, called positive control beads, bonded to IgGs,
polystyrene beads, called negative control beads, without a surface antigen. Such a laboratory kit (Labscreen Mixed®) comprises an aqueous suspension of polystyrene beads for screening anti-HLA class I antibodies and anti-HLA class II antibodies. The polystyrene beads in this suspension comprise a plurality of types of polystyrene beads, each type of polystyrene bead being distinguished from the other types of polystyrene bead by means of a fluorescent marker and comprising polystyrene beads carrying distinct HLA class I antigens and HLA class II antigens. In practice, each type of polystyrene bead has on the surface of the polystyrene beads up to six HLA-A (class I) antigens, six HLA-B (class I) antigens, six HLA-C (class I) antigens, or six HLA-DQ (class II) antigens, six HLA-DR (class II) antigens, six HLA-DP (class II) antigens.

The laboratory kit (Labscreen Mixed®) comprises 12 types of class I polystyrene beads and 5 types of class II polystyrene beads, the fluorescence intensity of each of the 17 types of polystyrene beads being able to be measured simultaneously. Accordingly, the average of the fluorescence intensity of each of the types of polystyrene beads bonded to the same group of HLA antigens is calculated. Within the mixture of polystyrene beads, some are without an HLA class I antigen or HLA class II antigen and serve as negative control.

The use of the laboratory kit (Labscreen Mixed®) additionally requires a negative control serum (LabScreen Negative Control (LSNC) serum) in the anti-HLA antibody screening reaction. The serum is characterized by the manufacturer as being without anti-HLA class I antibodies and anti-HLA class II antibodies.

The average values of the fluorescence intensity, observed over a period of five months, associated with each of the 12 types of beads carrying class I antigens and with each of the 5 types of beads carrying class II antigens treated with such a negative control are given in Table 1 below.

TABLE 1

| Class of HLA | | | |
|---|---|---|---|
| Ag | | | Standard |
| recognized | Bead number | Average fluorescence | deviation |
| Class I | 6 | 79.82 | 30.26 |
| | 7 | 110.87 | 39.45 |
| | 88 | 118.09 | 30.31 |
| | 17 | 94.16 | 38.98 |
| | 69 | 136.93 | 54.01 |
| | 79 | 136.67 | 49.73 |
| | 84 | 109.79 | 35.61 |
| | 86 | 102.07 | 27.77 |
| | 87 | 119.96 | 32.50 |
| | 88 | 100.67 | 30.04 |
| | 89 | 90.89 | 25.85 |
| | 90 | 157.12 | 73.60 |
| Class II | 91 | 112.79 | 35.91 |
| | 93 | 143.52 | 39.21 |
| | 95 | 104.89 | 34.79 |
| | 96 | 153.14 | 55.02 |
| | 97 | 117.61 | 46.14 |

The values presented in Table 1 are low and reflect the fluorescence generated by the non-specific binding of the IgGs to the polystyrene beads, and the non-specific binding of the secondary antibody to the beads.

Positive Control

The laboratory kit (Labscreen Mixed®) comprises, as positive control, polystyrene beads to the surface of which there are grafted purified human IgGs. Such a positive control is limited in its use to the verification of the functionality of the secondary antibody. Such a positive control does not allow a value of the fluorescence intensity to be converted into a value of the concentration of anti-HLA antibodies in a liquid medium.

Polyclonal Positive Control

The users of the kit (Labscreen Mixed®), especially immunology laboratories, recommend using, as positive control in the screening of anti-HLA antibodies in vitro according to the prior art, a mixture of serums collected from individuals polyimmunized against the HLA antigens. By way of example, the average values of the fluorescence intensity, measured over a period of 5 months, associated with each type of HLA class I and class II beads of this polyclonal control are given in Table 2 below.

TABLE 2

| Class of HLA | | | |
|---|---|---|---|
| Ag | | | Standard |
| recognized | Bead number | Average fluorescence | deviation |
| Class I | 6 | 3821.21 | 1980.68 |
| | 7 | 5313.44 | 2261.91 |
| | 88 | 4103.80 | 1812.89 |
| | 17 | 3808.23 | 1726.83 |
| | 69 | 2699.31 | 1759.92 |
| | 79 | 2316.98 | 1341.26 |
| | 84 | 2437.50 | 1672.73 |
| | 86 | 3163.40 | 1765.90 |
| | 87 | 1836.57 | 1379.55 |
| | 88 | 2082.32 | 1364.82 |
| | 89 | 15691.55 | 1791.89 |
| | 90 | 15158.40 | 2072.65 |
| Class II | 91 | 14433.92 | 2197.01 |
| | 93 | 13343.17 | 2492.20 |
| | 95 | 14550.62 | 2162.28 |
| | 96 | 207.17 | 232.44 |
| | 97 | 589.34 | 670.31 |

The calculated value of the average fluorescence measured on the HLA class I beads treated with a qualitative control according to the prior art is approximately 5200 fluorescence units, and the standard deviation is approximately 4900 fluorescence units.

The calculated value of the average fluorescence measured on the HLA class II beads treated with a qualitative control according to the prior art is approximately 8600 fluorescence units, and the standard deviation is approximately 7500 fluorescence units.

The fluorescence intensity of each of the types of polystyrene bead of the qualitative control varies between 1000 and 20,000. Such variability in the response of each of the types of polystyrene beads (HLA-A, HLA-B, HLA-C, HLA-DQ, HLA-DR and HLA-DP) does not allow a single curve for conversion of the measured fluorescence intensity into a reference anti-HLA antibody concentration to be defined. In addition, such a curve for conversion of the measured fluorescence intensity cannot be obtained, given the fact that the concentration of HLA antibodies specific to each of the types of HLA antigen cannot be determined in the qualitative control of the prior art.

By way of example of a qualitative control of the prior art, an analysis of the fluorescence of each type of polystyrene bead of the “Labscreen Mixed®” kit shows that the fluorescence associated with each type of bead carrying a class I antigen (bead type no. 6, 7, 8, 17, 69, 79, 84, 86, 87, 88, 89 and 90 in FIG. 4A) varies between a value of approximately 7000 average fluorescence units and 12,000 average fluorescence units. The average value of the fluorescence intensities measured on each type of HLA class I bead is approximately 9600 fluorescence units. The value of the standard deviation of these values is approximately 3100 fluorescence units.

In addition, this analysis of the fluorescence of each type of polystyrene bead of the “Labscreen Mixed®” kit shows that the fluorescence associated with each type of bead carrying a class II antigen (bead type no. 91, 93, 95, 96 and 97 in FIG. 4B) varies between a value of approximately 1000 average fluorescence units and 19,000 average fluorescence units. The average value of the fluorescence intensities measured on each type of HLA class II bead is approximately 13,000 fluorescence units. The value of the standard deviation of these values is approximately 8300 fluorescence units.

Such a control is therefore limited in its use to a purely qualitative analysis since the concentration of each antibody in the serum mixture is not known.

Monoclonal Chimeric Immunoglobulin According to the Invention

There is used, as quantitative control of the laboratory kit (Labscreen Mixed®), a monoclonal chimeric immunoglobulin according to the invention. Solutions of an anti-HLA class I monoclonal chimeric immunoglobulin (Hu-IgG1 K [W6/32]) and/or of an anti-HLA class II monoclonal chimeric immunoglobulin (Hu-IgG1 K [F3.3]) as described in Examples 1 to 4 at a known concentration of 2 μg/ml are prepared. An analysis of the average fluorescence intensity associated with each type of polystyrene bead carrying an HLA class I or class II antigen is carried out. The results obtained are presented in FIG. 4A and FIG. 4B (white histograms).

For each type of polystyrene bead of the “Labscreen Mixed®” kit, a fluorescence intensity of approximately 22,000 average fluorescence units is observed.

In particular, this analysis shows that:
the fluorescence associated with each type of bead carrying a class I antigen (bead type no. 6, 7, 8, 17, 69, 79, 84, 86, 87, 88, 89 and 90 in FIG. 4A, solid histograms) is substantially constant and is approximately 22,000 average fluorescence units. The average value of the fluorescence intensities measured on each type of HLA class I bead is approximately 21,500 fluorescence units. The value of the standard deviation of these values is approximately 450 fluorescence units;
the fluorescence associated with each type of bead carrying a class II antigen (bead type no. 91, 93, 95, 96 and 97 in FIG. 4B, solid histograms) is substantially constant and is approximately 22,000 average fluorescence units. The value of the standard deviation of these values is approximately 700 fluorescence units.

This fluorescence intensity value is substantially constant whatever the type of polystyrene bead (whatever the HLA antigen carried by the type of polystyrene bead) and corresponds to an antibody concentration of 2 μg/ml. Such chimeric monoclonal antibodies according to the invention are suitable for permitting a quantitative calibration of the fluorescence response as a function of the concentration of chimeric monoclonal antibodies according to the invention.

By way of comparison of the invention (solid histograms in FIGS. 4A and 4B) and of the prior art (hatched histograms in FIGS. 4A and 4B), the average fluorescence values (and standard deviation) found on the beads carrying HLA class I antigens and on the beads carrying HLA class II antigens are shown in FIG. 4A and FIG. 4B.

### EXAMPLE 6—PROCESS FOR THE IN VITRO QUANTIFICATION OF ANTI-HLA ANTIBODIES—“DOSE/RESPONSE” CURVES

In order to produce a “dose/response” curve, 300 μl of a washing buffer (PBS) are distributed in each of the wells of a multi-well microfilter plate (Multiscreen®). After 10 minutes, the washing buffer is removed by suction, and 5 μl of homogenized beads suspension “Labscreen Mixed®” (LSM12, One Lambda® Inc., USA, containing either 12 different types of beads grafted with various HLA-A, HLA-B and HLA-C purified HLA class I antigens or 5 different types of beads grafted with various HLA-DR, HLA-DQ and HLA-DP purified HLA class II antigens) are added. A range of solutions of chimeric monoclonal antibodies according to the invention of decreasing concentrations of chimeric monoclonal antibodies is obtained by successive serial dilutions. The negative and positive controls are treated in the same way in parallel. 20 μl of each of the serial dilutions are placed in contact with polystyrene beads which have previously been placed in the wells of the multi-well plate. The mixtures of polystyrene beads and chimeric monoclonal antibodies are incubated at ambient temperature and with the exclusion of light for 30 minutes. At the end of the incubation, each of the wells is washed five times in succession with 250 μl of a washing buffer. 100 μl of a solution of secondary antibody diluted to 1/100 are then added. The secondary antibodies used for the fluorescent labelling of the anti-HLA antibodies bound to the polystyrene beads are, for example, goat anti-IgA antibodies coupled to phycoerythrin (anti-IgA-PE, AbSerotec, USA) or goat anti-IgG antibodies coupled to phycoerythrin (anti-IgG-PE, InGen, USA). Of course, any other fluorescent group coupled to an antibody capable of recognizing and binding to the constant chains of the monoclonal chimeric immunoglobulin according to the invention may be used. The multi-well plates are placed away from the light and at ambient temperature for 30 minutes, before being analyzed in a Luminex® immunofluorescence reader.

Calibration curves of the “dose/response” type obtained by a process according to the invention are shown in FIGS. 6 (anti-HLA class I) and 7 (anti-HLA class II). The value of the fluorescence intensity is given as a function of the logarithm of the concentration of monoclonal chimeric immunoglobulin according to the invention expressed in μg/ml.

The twelve calibration curves (FIG. 6A) obtained with the twelve different types of beads grafted with different HLA class I antigens are well described by Boltzmann equations (R²>0.999). The twelve calibration curves are highly similar. The average fluorescence value associated to the twelve different types of beads for each dilution of the monoclonal chimeric immunoglobulin are used to calculate a single average calibration curve (FIG. 6B).

The five calibration curves (FIG. 7A) obtained with the twelve different types of beads grafted with different HLA class II antigens are well described by Boltzmann equations (R²>0.999). The five calibration curves are highly similar. The average fluorescence value associated to the five different types of beads for each dilution of the monoclonal chimeric immunoglobulin are used to calculate a single average calibration curve (FIG. 7B).

The average values and the standard deviations of the fluorescence intensity measurement measured on each type of HLA class I beads (FIG. 6B) and HLA class II beads (FIG. 7B) are summarized in Table 3 below.

TABLE 3

| | HLA class I | | HLA class II | |
|---|---|---|---|---|
| Antibody, | | Standard | | Standard |
| μg/ml | Average | deviation | Average | deviation |
| 5 × 10⁻⁴ | 589.25 | 31.15 | 316.30 | 29.6 |
| 2 × 10⁻³ | 1433.18 | 103.03 | 1086.38 | 72.15 |
| 7.8 × 10⁻³ | 3028.43 | 143.22 | 3518.68 | 145.09 |
| 3.13 × 10⁻² | 7781.63 | 342.02 | 9129.82 | 273.56 |
| 1.25 × 10⁻¹ | 15190.29 | 685.49 | 17955.55 | 298.79 |
| 5 × 10⁻¹ | 20548.53 | 441.7 | 22755.75 | 160.45 |
| 2 × 10⁰ | 22621.17 | 255.88 | 23578.19 | 167 |

A statistical analysis by sigmoid non-linear regression of the Boltzmann type (R²>0.999) is carried out on the data relating to the “dose/response” curves described above. There are determined the minimum observed fluorescence value (MIN), that is to say the value of the asymptote of the curve when the concentration of monoclonal chimeric immunoglobulin tends towards the value 0, the maximum observed fluorescence value (MAX), that is to say the value of the asymptote of the curve when the concentration of monoclonal chimeric immunoglobulin tends towards the infinite value, and the value of the concentration of monoclonal chimeric immunoglobulin corresponding to 50% of the specific signal (MAX-MIN). These values are given in Table 4 below.

TABLE 4

| Monoclonal chimeric | | | (MAX − MIN) * 50%, |
|---|---|---|---|
| immunoglobulin | MIN | MAX | μg/ml |
| Anti-HLA class I | 65 | 22400 | 0.06 |
| Anti-HLA class II | 88 | 24600 | 0.05 |

It is determined that the so-called threshold values of the fluorescence intensity value beyond which the concentration of monoclonal chimeric immunoglobulin are detected and considered as significantly present (non-zero quantity) in the serum are about 700 for both the anti-HLA class I antibodies and the anti-HLA class II antibodies.

### EXAMPLE 7—DETERMINATION OF THE QUANTITY OF ANTI-HLA CLASS I ANTIBODIES IN THE SERUM OF PATIENTS, USING THE “DOSE/RESPONSE” CURVES AS DESCRIBED IN EXAMPLE 6

It is proceeded with the serum of five patients in the same time and in the same conditions than for the production of the “dose/response” curve, wherein the serum of the patient is used in place of the solution of chimeric monoclonal antibodies according to the invention. Therefore, a volume of 300 μl of a washing buffer (PBS) is distributed in each of the wells of a multi-well microfilter plate)(Multiscreen®). After 10 minutes, the washing buffer is removed by suction, and 5 μl of homogenized polystyrene beads suspension “Labscreen Mixed®” are added in the wells.

A volume of 20 μl of the serum obtained from the blood of a patient is placed in contact with the polystyrene beads suspension in the well of the multi-well plate. The mixtures of polystyrene beads and serum containing antibodies are incubated at ambient temperature and with the exclusion of light for 30 minutes. At the end of the incubation, each of the wells is washed five times in succession with 250 μl of a washing buffer. 100 μl of a solution of the secondary antibody diluted to 1/100 are then added. The secondary antibodies used for the fluorescent labelling of the anti-HLA antibodies present in the serum and bound to the polystyrene beads are, for example, goat anti-IgA antibodies coupled to phycoerythrin (anti-IgA-PE, AbSerotec, USA) or goat anti-IgG antibodies coupled to phycoerythrin (anti-IgG-PE, InGen, USA) as mentioned in the example 6. The multi-well plates are placed away from the light and at ambient temperature for 30 minutes, before being analyzed in a Luminex® immunofluorescence reader.

Anti-HLA Class I Antibodies

The fluorescence intensities associated with each type of polystyrene beads grafted with HLA class I antigens are reported in table 5 below together with the HLA Class I alleles grafted on each type of polystyrene beads according to the HLA nomenclature.

TABLE 5

| HLA class I alleles grafted on each | Fluorescence intensity | | | | |
|---|---|---|---|---|---|
| type of beads | Patient 1 | Patient 2 | Patient 3 | Patient 4 | Patient 5 |
| A*01:01, A*29:02, B*08:01, | 166.03 | 804.11 | 1660.56 | 905.28 | 683.68 |
| B*45:01, C*06:02, C*07:01 | | | | | |
| A*02:01, A*31:01, B*15:01, | 207.23 | 157.29 | 251.42 | 1901.08 | 769.78 |
| B*67:01, C*07:02, — | | | | | |
| A*02:06, A*80:01, B*18:01, | 275.99 | 195.42 | 210.58 | 1930.28 | 1336.09 |
| B*27:05, C*02:02, — | | | | | |
| A*02, —, B*40:01/55, B*56:01/40, | 186.18 | 126.4 | 275.17 | 2157.39 | 472.02 |
| C*01:02, C*03:04 | | | | | |
| A*02:01, —, B*52:01, B*73:01, | 255.6 | 166.08 | 158.57 | 2279.35 | 1012.04 |
| C*07:01, C*15:05 | | | | | |
| A*11:01, A*30:01, B*13:02, | 148.6 | 167.67 | 161.48 | 3980.86 | 1656.44 |
| B*15:02, C*06:02, C*08:01 | | | | | |
| A*33:03, A*68:02/34, B*37:01, | 144.96 | 131.07 | 213.3 | 1915.28 | 503.1 |
| B*58:01, C*02:02/10, C*07:01/18 | | | | | |
| A*03:01, A*32:01, B*40:02, | 259.86 | 144.2 | 174.48 | 960.32 | 1046.72 |
| B*47:01, C*02:02/09/11, C*06:02 | | | | | |
| A*03:01/14/20/21N, A*66:01, | 175.88 | 163.94 | 176.46 | 768.88 | 928.58 |
| B*15:03, B*52:01, C*02:10, | | | | | |
| C*12:02 | | | | | |
| A*26:01, A*34:01, B*38:02, | 174.23 | 157.24 | 443.89 | 1154.45 | 855 |
| B*15:21, C*04:03, C*07:02 | | | | | |
| A*29:02, A*74:01, B*13:02, | 170.88 | 171.5 | 209.38 | 610.91 | 2178.41 |
| B*44:03, C*06:02, C*16:01/08 | | | | | |
| A*30:01, A*33:03, B*53:01, | 164.25 | 141.75 | 193.82 | 853.39 | 674.46 |
| B*81:01, C*04:01, C*08:04 | | | | | |

The logarithm of the concentration of the anti-HLA class I antibodies in the serum of the patient is deduced from theses measured values of fluorescence intensity as compared to the “dose/response” curves of example 6 (FIGS. 6A and 6B) and the logarithm of the concentration converted into the concentration values of antibodies. These concentrations are those of chimeric antibodies giving the same signal than the serum of the considered patient. The deduced values of the concentration of anti-HLA class I antibodies present in the different serum are reported in table 6 below together with the HLA Class I alleles grafted on each type of polystyrene beads according to the HLA nomenclature.

TABLE 6

| HLA alleles grafted on a type of | Antibody concentration, ng/mL | | | | |
|---|---|---|---|---|---|
| beads | Patient 1 | Patient 2 | Patient 3 | Patient 4 | Patient 5 |
| A*01:01, A*29:02, B*08:01, | LTT | 0.68 | 3.17 | 0.95 | LTT |
| B*45:01, C*06:02, C*07:01 | | | | | |
| A*02:01, A*31:01, B*15:01, | LTT | LTT | LTT | 3.92 | 0.59 |
| B*67:01, C*07:02, — | | | | | |
| A*02:06, A*80:01, B*18:01, | LTT | LTT | LTT | 4.01 | 2.19 |
| B*27:05, C*02:02, — | | | | | |
| A*02, —, B*40:01/55, B*56:01/40, | LTT | LTT | LTT | 4.75 | LTT |
| C*01:02, C*03:04 | | | | | |
| A*02:01, —, B*52:01, B*73:01, | LTT | LTT | LTT | 5.15 | 1.25 |
| C*07:01, C*15:05 | | | | | |
| A*11:01, A*30:01, B*13:02, | LTT | LTT | LTT | 11.43 | 3.15 |
| B*15:02, C*06:02, C*08:01 | | | | | |
| A*33:03, A*68:02/34, B*37:01, | LTT | LTT | LTT | 3.97 | LTT |
| B*58:01, C*02:02/10, C*07:01/18 | | | | | |
| A*03:01, A*32:01, B*40:02, | LTT | LTT | LTT | 1.11 | 1.35 |
| B*47:01, C*02:02/09/11, C*06:02 | | | | | |
| A*03:01/14/20/21N, A*66:01, | LTT | LTT | LTT | 0.58 | 1.02 |
| B*15:03, B*52:01, C*02:10, | | | | | |
| C*12:02 | | | | | |
| A*26:01, A*34:01, B*38:02, | LTT | LTT | LTT | 1.66 | 0.82 |
| B*15:21, C*04:03, C*07:02 | | | | | |
| A*29:02, A*74:01, B*13:02, | LTT | LTT | LTT | LTT | 4.82 |
| B*44:03, C*06:02, C*16:01/08 | | | | | |
| A*30:01, A*33:03, B*53:01, | LTT | LTT | LTT | 0.81 | LTT |
| B*81:01, C*04:01, C*08:04 | | | | | |

Low signals of fluorescence observed with certain patients were lower that the threshold of detection as indicated in the table 6 as “LTT”. The process according to the invention allows to determine the concentration or the absence of anti-HLA antibody in the serum of patients.

Anti-HLA Class II Antibodies

The fluorescence intensities associated with each type of polystyrene beads grafted with HLA class II antigens are reported in table 7 below together with the HLA class II alleles grafted on each type of polystyrene beads according to the HLA nomenclature.

TABLE 7

| HLA class II alleles grafted on each type | Fluorescence intensity | | | | |
|---|---|---|---|---|---|
| of beads | Patient 1 | Patient 2 | Patient 3 | Patient 4 | Patient 5 |
| DRB1*01:02, DRB1*11:01, —, | 3933.43 | 134.29 | 788.87 | 9289.61 | 241.32 |
| DRB3*02:02, DQA1*01:01, DQA1*01:02, | | | | | |
| DQB1*05:01, DQB1*06:02, DPA1*02:01, | | | | | |
| DPA1*03:01, DPB1*01:01, DPB1*40:01 | | | | | |
| DRB1*01:03, DRB1*03:01, —, | 8294.52 | 178.2 | 512.15 | 4512.57 | 437.64 |
| DRB3*02:02, DQA1*01:01, DQA1*05:01, | | | | | |
| DQB1*02:01, DQB1*05:01, DPA1*01:03, | | | | | |
| DPA1*02:01, DPB1*02:01, DPB1*106:01 | | | | | |
| DRB1*03:02, DRB1*12:01, DRB3*01:01, | 746.71 | 169.18 | 1630.26 | 1779.46 | 240.2 |
| —, DQA1*01:05, DQA1*04:01, | | | | | |
| DQB1*04:02, DQB1*05:01, DPA1*01:03, | | | | | |
| DPA1*02:02, DPB1*01:01, DPB1*18:01 | | | | | |
| DRB1*13:03, DRB1*16:01, DRB3*01:01, | 5949.01 | 211.8 | 849.23 | 2148.12 | 242.23 |
| DRB5*02:02, DQA1*01:02, | | | | | |
| DQA1*05:05/09, DQB1*03:01, | | | | | |
| DQB1*05:02, DPA1*01:03, DPA1*02:02, | | | | | |
| DPB1*01:01, DPB1*02:02 | | | | | |
| DRB1*13:01, DRB1*14:54, DRB3*01:01, | 4412.37 | 180.35 | 1193.91 | 2021.27 | 282.21 |
| DRB3*02:02, DQA1*01:03, DQA1*01:04, | | | | | |
| DQB1*05:03, DQB1*06:03, DPA1*02:02, | | | | | |
| —, DPB1*01:01, DPB1*05:01 | | | | | |

The logarithm of the concentration of the anti-HLA class II antibodies in the serum of the patient is deduced from theses measured values of fluorescence intensity as compared to the “dose/response” curves of example 6 (FIGS. 7A and 7B) and the logarithm of the concentration converted into the concentration values of antibodies. These concentrations are those of chimeric antibodies giving the same signal than the serum of the considered patient. The deduced values of the concentration of anti-HLA class II antibodies present in the different serum are reported in table 8 below.

TABLE 8

| HLA class II alleles grafted on each type | Antibody concentration, ng/mL | | | | |
|---|---|---|---|---|---|
| of beads | Patient 1 | Patient 2 | Patient 3 | Patient 4 | Patient 5 |
| DRB1*01:02, DRB1*11:01, —, DRB3*02:02, | 10.08 | LTT | 1.23 | 30.78 | LTT |
| DQA1*01:01, DQA1*01:02, DQB1*05:01, | | | | | |
| DQB1*06:02, DPA1*02:01, DPA1*03:01, | | | | | |
| DPB1*01:01, DPB1*40:01 | | | | | |
| DRB1*01:03, DRB1*03:01, —, DRB3*02:02, | 26.06 | LTT | LTT | 11.87 | LTT |
| DQA1*01:01, DQA1*05:01, DQB1*02:01, | | | | | |
| DQB1*05:01, DPA1*01:03, DPA1*02:01, | | | | | |
| DPB1*02:01, DPB1*106:01 | | | | | |
| DRB1*03:02, DRB1*12:01, DRB3*01:01, —, | 1.11 | LTT | 3.52 | 3.93 | LTT |
| DQA1*01:05, DQA1*04:01, DQB1*04:02, | | | | | |
| DQB1*05:01, DPA1*01:03, DPA1*02:02, | | | | | |
| DPB1*01:01, DPB1*18:01 | | | | | |
| DRB1*13:03, DRB1*16:01, DRB3*01:01, | 16.69 | LTT | 1.40 | 4.94 | LTT |
| DRB5*02:02, DQA1*01:02, | | | | | |
| DQA1*05:05/09, DQB1*03:01, | | | | | |
| DQB1*05:02, DPA1*01:03, DPA1*02:02, | | | | | |
| DPB1*01:01, DPB1*02:02 | | | | | |
| DRB1*13:01, DRB1*14:54, DRB3*01:01, | 11.55 | LTT | 2.34 | 4.59 | LTT |
| DRB3*02:02, DQA1*01:03, DQA1*01:04, | | | | | |
| DQB1*05:03, DQB1*06:03, DPA1*02:02, —, | | | | | |
| DPB1*01:01, DPB1*05:01 | | | | | |

Low signals of fluorescence observed with certain patients were lower that the threshold of detection as indicated in the table 8 as “LTT”.

### EXAMPLE 8—ANALYSIS OF THE STABILITY OF THE MONOCLONAL CHIMERIC IMMUNOGLOBULINS HU-IgG1 K [W6/32] AND HU-IgG1 K [F3.3] ACCORDING TO THE INVENTION IN AN ALBUMINOUS BUFFER MEDIUM

Serial dilutions of the monoclonal chimeric immunoglobulins according to the invention stored for a period of two months in an aqueous buffer medium containing human albumin at a concentration of 60 g/l are prepared. “Dose/response” curves as described in Example 6 are prepared by the Luminex® technique. The results obtained with the monoclonal chimeric immunoglobulin directed against the HLA class I antigens (Hu-IgG1 K [W6/32]) are presented in FIG. 8A and the results obtained with the monoclonal chimeric immunoglobulin directed against the HLA class II antigens (Hu-IgG1 K [F3.3]) are presented in FIG. 8B. The results obtained with the monoclonal chimeric immunoglobulins stored in the saline buffer are identified by white squares (□) and the results obtained with the monoclonal chimeric immunoglobulins stored in the albuminous buffer are identified by black lozenges (♦). No significant difference is observed between the monoclonal chimeric immunoglobulins stored in saline buffer and the monoclonal chimeric immunoglobulins stored in albuminous buffer.

1. Process for determining the quantity of anti-HLA antibodies in a liquid medium susceptible to contain antibodies, the process comprising:
determining a calibration curve, said calibration curve being defined by a plurality of measured values of a parameter, each measured value (Vₙ) of the plurality of values corresponding to a determined quantity (Qₙ) of a monoclonal chimeric immunoglobulin according to the invention;
measuring a value (Vₓ) of the parameter corresponding to the liquid medium, and
deducing the quantity of anti-HLA antibody in the liquid medium from the calibration curve and from the measured value (Vₓ) of the parameter corresponding to the liquid medium;
wherein the monoclonal chimeric immunoglobulin consists of:
two polypeptide heavy chains (H), of molecular weight from 40 kDa to 60 kDa, and
two polypeptide light chains (L), of molecular weight from 20 kDa to 30 kDa,
wherein:
each heavy chain (H) comprises:
a heavy chain variable region (V_{H}) of a monoclonal antibody selected from the group consisting of monoclonal antibodies specific to monomorphic epitopes of HLA class I antigens and monoclonal antibodies specific to monomorphic epitopes of HLA class II antigens, and
a heavy chain constant region (C_{H}) of a human immunoglobulin selected from the group consisting of IgAs, IgGs and IgMs,
and wherein:
each light chain (L) comprises:
a light chain variable region (V_{L}) of a monoclonal antibody selected from the group consisting of monoclonal antibodies specific to monomorphic epitopes of HLA class I antigens and monoclonal antibodies specific to monomorphic epitopes of HLA class II antigens, and
a light chain constant region (C_{L}) of a human immunoglobulin selected from the group consisting of the kappa chains and the lambda chains.

2. The process of claim 1, wherein the liquid medium is a biological fluid of a patient.

3. The process of claim 1, wherein determining the calibration curve comprises:
preparing a plurality of solutions (Sₙ), especially aqueous solutions, of a monoclonal chimeric immunoglobulin, each solution (Sₙ) comprising a determined quantity (Qₙ) of said monoclonal chimeric immunoglobulin and having a determined concentration value (Cₙ) of said monoclonal chimeric immunoglobulin, and then
placing a predetermined volume of each solution (Sₙ) in contact with a same determined quantity of at least one immobilized HLA antigen, and
measuring a value, named measured value (Vₙ), of a parameter, said measured value (Vₙ) being related to the quantity of the monoclonal chimeric immunoglobulin bound to the defined quantity of each immobilized HLA antigen,
forming pairs (Cₙ, Vₙ) of the defined concentration (Cₙ) and the measured value (Vₙ) representing the variation of the measured value (Vₙ) as a function of the defined concentration (Cₙ) of monoclonal chimeric immunoglobulin of each solution (Sₙ) of monoclonal chimeric immunoglobulin (dose/response data),
carrying out a statistical analysis—especially by sigmoid non-linear regression of the Boltzmann type—of the (Cₙ, Vₙ) pairs and determining the calibration curve by this statistical analysis.

4. The process of claim 3, wherein from the calibration curve there is calculated a value, called a threshold value, of the parameter beyond which the concentration of monoclonal chimeric immunoglobulin is significantly greater than 0.

5. The process of claim 1, wherein the light chain variable region (V_{L}) of the monoclonal chimeric immunoglobulin is a light chain variable region of a monoclonal antibody of a vertebrate and the heavy chain variable region (V_{H}) of the monoclonal chimeric immunoglobulin is a heavy variable region of a heavy chain variable region of a monoclonal antibody of a vertebrate.

6. The process of claim 1, wherein the parameter is selected from the group consisting of a fluorescence parameter, a luminescence parameter and a colorimetry parameter.

7. The process of claim 6, wherein the fluorescence parameter is a fluorescence intensity.

8. The process of claim 1, wherein:
a) each immobilized HLA antigen is an HLA antigen immobilized on the surface of particles of a solid substrate in the divided state, said solid substrate being formed of particles,
b) the immobilized HLA antigens and each solution of monoclonal chimeric immunoglobulin directed against the HLA antigens of the solid substrate are brought into contact under conditions suitable for stable bonding between the HLA antigens of the solid substrate and the monoclonal chimeric immunoglobulin of each solution of monoclonal chimeric immunoglobulin, and then
c) the monoclonal chimeric immunoglobulins that are not bound to the HLA antigens of the solid substrate are removed by washing, and then
d) the monoclonal chimeric immunoglobulins that are bound to the HLA antigens of the solid substrate are brought into contact with a solution of a secondary antibody which is selected from the group consisting of fluorescent secondary antibodies, luminescent secondary antibodies and photoabsorbent secondary antibodies and which is directed against the monoclonal chimeric immunoglobulin, under conditions suitable for stable bonding between the monoclonal chimeric immunoglobulin and the secondary antibody, and then
e) the secondary antibody that is not bound to the monoclonal chimeric immunoglobulin is removed by washing, and then
f) at least one parameter of the secondary antibody that is bound to each particle of the solid substrate is measured, and there is assigned to that measurement a measured value (Vₙ) of said parameter selected from the group consisting of a fluorescence parameter, a luminescence parameter and a colorimetry parameter, and then
g) the calibration curve is formed.

9. The process of claim 8, wherein:
h) there is derived from the calibration curve a fluorescence intensity threshold value indicating the presence of the anti-HLA antibody in a solution to be analyzed.

10. The process of claim 1, wherein each immobilized HLA antigen is an HLA antigen presented at the surface of at least one cell.

11. The process of claim 1, wherein the light chain variable region (V_{L}) of the monoclonal chimeric immunoglobulin is the light chain variable region of the W6/32 antibody and the heavy chain variable region (V_{H}) of the monoclonal chimeric immunoglobulin is the heavy chain variable region of the W6/32 antibody.

12. The process of claim 1, wherein the light chain variable region (V_{L}) of the monoclonal chimeric immunoglobulin is the light chain variable region of the F3.3 antibody and the heavy chain variable region (V_{H}) of the monoclonal chimeric immunoglobulin is the heavy chain variable region of the F3.3 antibody.

13. The process of claim 1, wherein the monoclonal chimeric immunoglobulin is a monoclonal chimeric immunoglobulin specific to HLA class I antigens selected from the group consisting of:
monoclonal chimeric immunoglobulins comprising at least one light chain of sequence SEQ ID_NO 1, and
monoclonal chimeric immunoglobulins comprising at least one heavy chain chosen from the group formed of heavy chains of sequence SEQ ID_NO 2, heavy chains of sequence SEQ ID_NO 3 and heavy chains of sequence SEQ ID_NO 4.

14. The process of claim 1, wherein the monoclonal chimeric immunoglobulin specific is a monoclonal chimeric immunoglobulin to HLA class II antigens selected from the group consisting of:
monoclonal chimeric immunoglobulins comprising at least one light chain of sequence SEQ ID_NO 5, and
monoclonal chimeric immunoglobulins comprising at least one heavy chain chosen from the group formed of heavy chain of sequence SEQ ID_NO 6, heavy chain of sequence SEQ ID_NO 7 and heavy chain of sequence SEQ ID_NO 8.


SEQUENCE LISTING (8 sequences)

SEQ ID NO: 1 - protein, 235 aa, Mus
  SOURCE 1..235: /mol_type=“protein” /note=“SEQ ID_NO 1 - Chaine legere de Hu-IgG1 K [W6/32]” /organism=“Mus <mouse, genus>”
MKSQTQVFVFLLLCVSGAHGSIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPPWTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 469 aa, Mus
  SOURCE 1..469: /mol_type=“protein” /note=“SEQ ID_NO 2 - Chaine lourde de Hu-IgG1 K [W6/32]” /organism=“Mus <mouse, genus>”
MAVLVLLFCLVTFPSCVLSQVQLKQSGPGLVQPSQSLSLTCTVSGFSLTSYGVHWVRQPPGKGLEWLGVIWSGGSTDYNAAFISRLSIRKDNSKSQVFFKMNSLQADDTAIYYCARTFTTSTSAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 479 aa, Mus
  SOURCE 1..479: /mol_type=“protein” /note=“SEQ ID_NO 3 - Chaine lourde de Hu-IgA2 K [W6/32]” /organism=“Mus <mouse, genus>”
MAVLVLLFCLVTFPSCVLSQVQLKQSGPGLVQPSQSLSLTCTVSGFSLTSYGVHWVRQPPGKGLEWLGVIWSGGSTDYNAAFISRLSIRKDNSKSQVFFKMNSLQADDTAIYYCARTFTTSTSAWFAYWGQGTLVTVSAASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 590 aa, Mus
  SOURCE 1..590: /mol_type=“protein” /note=“SEQ ID_NO 4 - Chaine lourde de Hu-IgM K [W6/32]” /organism=“Mus <mouse, genus>”
MAVLVLLFCLVTFPSCVLSQVQLKQSGPGLVQPSQSLSLTCTVSGFSLTSYGVHWVRQPPGKGLEWLGVIWSGGSTDYNAAFISRLSIRKDNSKSQVFFKMNSLQADDTAIYYCARTFTTSTSAWFAYWGQGTLVTVSAASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 5 - protein, 235 aa, Mus
  SOURCE 1..235: /mol_type=“protein” /note=“SEQ ID_NO 5 - Chaine legere de Hu-IgG1 K [F3.3]” /organism=“Mus <mouse, genus>”
MDFQVQIFSFLLISASVILSRGQIVLTQSPAIMSASPGEKVTMTCSASSIVRYMYWFQQKPGSSPRLLIYDTSSLSSGVPVRFSGSGSGTSYSLTISRMEAEDAATYFCQQWSSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 477 aa, Mus
  SOURCE 1..477: /mol_type=“protein” /note=“SEQ ID_NO 6 - Chaine lourde de Hu-IgA2 K [F3.3]” /organism=“Mus <mouse, genus>”
MDLRLSCAFIIVLLKGVQSEVKLEESGGGLVQPGGSMKLSCVASGFTFSNSWMNWVRQSPEKGLEWVAEIRLKSNNYATRYAESVKGRFTISRDDSKSSVYLQMNNLRSEDTAIYYCTPLSYSFDYWGQGTTVTVSTASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 7 - protein, 467 aa, Mus
  SOURCE 1..467: /mol_type=“protein” /note=“SEQ ID_NO 7 - Chaine lourde de Hu-IgG1 K [F3.3]” /organism=“Mus <mouse, genus>”
MDLRLSCAFIIVLLKGVQSEVKLEESGGGLVQPGGSMKLSCVASGFTFSNSWMNWVRQSPEKGLEWVAEIRLKSNNYATRYAESVKGRFTISRDDSKSSVYLQMNNLRSEDTAIYYCTPLSYSFDYWGQGTTVTVSTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 588 aa, Mus
  SOURCE 1..588: /mol_type=“protein” /note=“SEQ ID_NO 8 - Chaine lourde de Hu-IgM K [F3.3]” /organism=“Mus <mouse, genus>”
MDLRLSCAFIIVLLKGVQSEVKLEESGGGLVQPGGSMKLSCVASGFTFSNSWMNWVRQSPEKGLEWVAEIRLKSNNYATRYAESVKGRFTISRDDSKSSVYLQMNNLRSEDTAIYYCTPLSYSFDYWGQGTTVTVSTASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY